(19) United States
(12) Patent Application Publication
(10) Pub. No.: US 2023/0024685 A1
(43) Pub. Date: Jan. 26, 2023

(54) TEST CARD FOR AGGLUTINATION ASSAY TO BE ASSESSED WITH COMPUTER-IMPLEMENTED IMAGE ANALYSIS

(71) Applicants: Techcyte, Inc., Orem, UT (US); NanoSpot.AI, Inc., Salt Lake City, UT (US)

(72) Inventors: Benjamin Cahoon, Orem, UT (US); Richard Smith, Orem, UT (US); Shane Swenson, Orem, UT (US); Bryan Worthen, Orem, UT (US); Russ Zimmerman, Orem, UT (US); Vanessa Redecke, Salt Lake City, UT (US); Hans Haecker, Salt Lake City, UT (US); Mark Astill, Centerville, UT (US); Rian Wendling, Salt Lake City, UT (US)

(73) Assignees: Techcyte, Inc., Orem, UT (US); NanoSpot.AI, Inc., Salt Lake City, UT (US)

(21) Appl. No.: 17/813,003

(22) Filed: Jul. 15, 2022

Related U.S. Application Data
(60) Provisional application No. 63/222,338, filed on Jul. 15, 2021.

(51) Int. Cl. G01N 33/53; G01N 33/543; G01N 33/80; G01N 33/569; G06T 7/00

(52) U.S. Cl. CPC G01N 33/5304; G01N 33/54306; G01N 33/80; G01N 33/56983; G06T 7/0014; G01N 2021/825

(57) ABSTRACT

Test cards for agglutination assays, wherein the test cards are configured for computer-implemented image analysis. A test card includes a negative control test region for receiving a negative control fluid sample, a positive control test region for receiving a positive control fluid sample, and a test sample region for receiving a test fluid sample. The test card includes one or more unique scannable codes comprising data for instructing a processor to capture an image of the test card that is suitable for computer-implemented image analysis.

### CROSS-REFERENCE TO RELATED APPLICATIONS

This application claims the benefit of U.S. Provisional Patent Application No. 63/222,338, filed Jul. 15, 2021, entitled “IMAGE ANALYSIS FOR CLASSIFYING HEMAGGLUTINATION AND QUANTIFYING PRESENCE OF ANTIBODIES,” which is incorporated herein by reference in its entirety, including but not limited to those portions that specifically appear hereinafter, the incorporation by reference being made with the following exception: In the event that any portion of the above-referenced provisional application is inconsistent with this application, this application supersedes the above-referenced provisional application.

### TECHNICAL FIELD

The present disclosure relates to image analysis and particularly relates to classifying blood agglutination with machine learning implementations.

### BACKGROUND

Agglutination is the clumping of particles, and this process may occur when an antigen is mixed with its corresponding antibody. Blood agglutination (hemagglutination) is a specific form of agglutination that involves red blood cells (RBCs). Hemagglutination has common uses in the laboratory, including for blood typing and the quantification of antigen dilutions in a hemagglutination assay. A hemagglutination assay can be implemented to determine whether a person is currently infected with an antigen, has been infected with an antigen in the past, and in some cases, whether the person has been vaccinated against the antigen.

Many antigens attach to molecules present on the surface of RBCs. A consequence of this is that at certain concentrations, an antigen suspension may bind together (agglutinate) the RBCs, thus preventing them from settling out of suspension. Because agglutination is not linked to infectivity, attenuated antigens can therefore be used in assays while an additional assay such as a plaque assay must be used to determine infectivity. By serially diluting an antigen suspension into an assay tray (a series of wells of uniform volume) and adding a standard amount of blood cells, an estimation of the number of antigen particles can be made.

One example of an antigen that can be quantified with a hemagglutination assay is SARS-CoV-2, or the virus responsible for the COVID-19 pandemic. While initial countermeasures were restricted to therapeutic interventions for those with severe infections and prophylactic social distancing, recent clinical studies demonstrated efficacy for various SARS-CoV-2 vaccines, whose deployment is underway in many countries. Still, proper pandemic management hinges on the availability of diagnostic tests, including those revealing the presence of antibodies (AB) against the virus. On a population scale, results obtained by these tests reveal seroprevalence and the dynamics of virus spread, helping to predict susceptibility of a population and guiding health care decisions. On an individual level, such test results indicate previous SARS-CoV-2 infection or successful vaccination and have high predictive value for disease susceptibility. While numerous AB tests are available, they do have certain limitations. Tests performed in reference laboratories, such as enzyme-linked immunosorbent assays (ELISA) and related technologies, require special equipment, are expensive, and typically associated with long turnaround times from sample acquisition to results. Point-of-care (POC) tests, like the lateral flow immuno-assay (LFIA or LFA), can be conducted with minimal laboratory equipment, but typically show reduced sensitivity compared to tests conducted in reference labs and are also relatively expensive. Moreover, analysis is performed by visual inspection, which is subjective in nature and thus affects specificity and sensitivity dependent on conservative interpretation. An alternative format for detection of antigen-specific antibodies in whole blood are hemagglutination tests (HAT). This format is typically based on bispecific proteins with one moiety, e.g., an AB, binding to red blood cells (RBC) and the other moiety encompassing the antigen, e.g., a virus protein as target for the investigated AB. Antigen-specific AB contained in whole blood bind to the test antigen and mediate visible agglutination of RBC within seconds to minutes.

However, such hemagglutination tests are historically assessed and quantifying using human-based visualization of samples. This is time consuming and introduces the risk of inaccurate assessments. It is therefore desirable to introduce systems and methods for efficient and accurate analysis of hemagglutination tests.

Considering the foregoing, disclosed herein are systems, methods, and devices for computer-based analysis of hemagglutination assays. The image analysis systems described herein execute linear scale quantification of hemagglutination assays to identify the presence of certain antibodies.

### BRIEF DESCRIPTION OF THE DRAWINGS

Non-limiting and non-exhaustive implementations of the disclosure are described with reference to the following figures, wherein like reference numerals refer to like parts throughout the various views unless otherwise specified. It will be appreciated by those of ordinary skill in the art that the various drawings are for illustrative purposes only. The nature of the present disclosure, as well as other embodiments in accordance with this disclosure, may be more clearly understood by reference to the following detailed description, to the appended claims, and to the several drawings.

FIG. 1 is a schematic diagram of a system for remote image analysis for classifying agglutination of fluid samples;

FIG. 2A is a schematic diagram of a system for data storage and analysis on a cloud-based storage system with fault-tolerant servers;

FIG. 2B is a schematic diagram of a system and process flow for accessing a cloud-based database;

FIG. 3A is a schematic illustration of red blood cells and antibodies in the presence of an antigen, wherein a portion of the antibodies do not interact with the antigen;

FIG. 3B is a schematic illustration of red blood cells and antibodies in the presence of an antigen, wherein the antibodies bind to the antigen and cause the red blood cells to agglutinate;

FIG. 4 is a schematic illustration of an agglutination test card;

FIG. 5 is a schematic diagram of a training methodology for training a machine learning algorithm on a dataset comprising samples from infected, non-infected, vaccinated, and non-vaccinated individuals;

FIG. 6 is a schematic illustration of a system for consistently capturing images of hemagglutination test cards for increasing accuracy of image analysis implemented by a machine learning algorithm;

FIG. 7 is a schematic diagram of a system for protecting information by partitioning data across multiple storage resources;

FIG. 8 is a schematic diagram of a system and process flow for communication with a data analysis server when capturing images of test cards or exchanging data with the data analysis server;

FIG. 9 is a schematic flow chart diagram of a method for computer-implemented image analysis of an agglutination assay;

FIG. 10 is a schematic flow chart diagram of a method for classifying agglutination of a blood sample;

FIG. 11 is a schematic flow chart diagram of a method for remote image analysis for visually assessing agglutination of fluid samples;

FIG. 12 is a schematic flow chart diagram of a method for protecting information by partitioning data across multiple storage resources; and

FIG. 13 is a schematic diagram of an example computing system.

### DETAILED DESCRIPTION

Disclosed herein are systems, methods, and devices for remote image analysis of agglutination assay test cards. Specifically disclosed herein are test cards designed for agglutination assays, wherein the test cards include unique scannable codes to aid in capturing images of the test card that are suitable for computer-implemented image analysis. Further specifically disclosed herein are systems, methods, and devices for computer-implemented image analysis of agglutination assay test cards. Further specifically disclosed herein are systems, methods, and devices for protecting patient information by partitioning data across multiple storage resources.

The systems, methods, and devices disclosed herein may be implemented for detecting antibodies based on hemagglutination. The image analysis described herein may specifically be implemented in connection with a hemagglutination-based antibodies test (HAT) that is based on bispecific proteins containing a dromedary-derived antibody (nanobody) that binds red blood cells (RBCs). In a specific implementation, the image analysis system is implemented in connection with a hemagglutination-based antibodies test for quantifying the presence of a certain antigen, such as a virus, bacteria, or other foreign agent within a patient's body.

Hemagglutination tests are typically interpreted by visual inspection, which is subjective and can be affected by the training and expertise of the individual interpreting the test. Described herein are systems, methods, and devices for computer-based processing that implements an image-based deep learning algorithm trained to identify and interpret hemagglutination on testing boards. In an implementation, a user uploads an image of an agglutination test image, which is then uploaded to a remote server for analysis and interpretation by a machine learning algorithm.

The machine learning algorithm described herein detects and classifies agglutinated and non-agglutinated blood spots in images captured by a camera. The deep learning object detection model is trained with various degrees of positive (agglutinated) and negative (non-agglutinated) labels, with additional positive and negative labels reserved for training validation. The model is configured to identify the location and type (i.e., degree of agglutination) of each blood spot in a captured image along with a confidence score in the interval, [0, 1], for each prediction. The performance of the resulting deep learning model is evaluated against a holdout test set composed of additional positive and negative image captures that had never been shown to the model in training. The machine learning algorithm is trained to classify the agglutination of the blood samples on both a quantitative and a qualitative scale.

In studies performed on the deep learning model described herein, the visual and computer-based test interpretation exhibited perfect correlation. The deep learning model correctly classified visually positive and negative samples. As such, an image-based, computer-driven approach can be used, in principle, to identify hemagglutination on cardboards with high confidence.

The systems, methods, and devices disclosed herein further include diagnostic tests for detecting antibodies against SARS-CoV-2. These systems and methods are important tools for managing the COVID-19 pandemic. Traditional antibody tests are available but have certain limitations regarding turnaround time, costs, and availability, particularly in point-of-care (POC) settings. The systems, methods, and devices disclosed herein enable quick and accurate testing that can be implemented in a point-of-care setting. For the assay described herein, two principal modes of test readout are possible, including visual inspection by a human and image/computer-based test analysis. Both take advantage of controls, which provide direct comparatives. While those may be less important for strong agglutination reactions, for example, upon vaccination, the negative control can be important to differentiate weak positive reactions from subtle, spontaneous agglutination found in some patients.

A detailed description of systems and methods consistent with embodiments of the present disclosure is provided below. While several embodiments are described, this disclosure is not limited to any one embodiment, but instead encompasses numerous alternatives, modifications, and equivalents. In addition, while numerous specific details are set forth in the following description to provide a thorough understanding of the embodiments disclosed herein, some embodiments may be practiced without some or all these details. Moreover, for the purpose of clarity, certain technical material that is known in the related art has not been described in detail to avoid unnecessarily obscuring the disclosure.

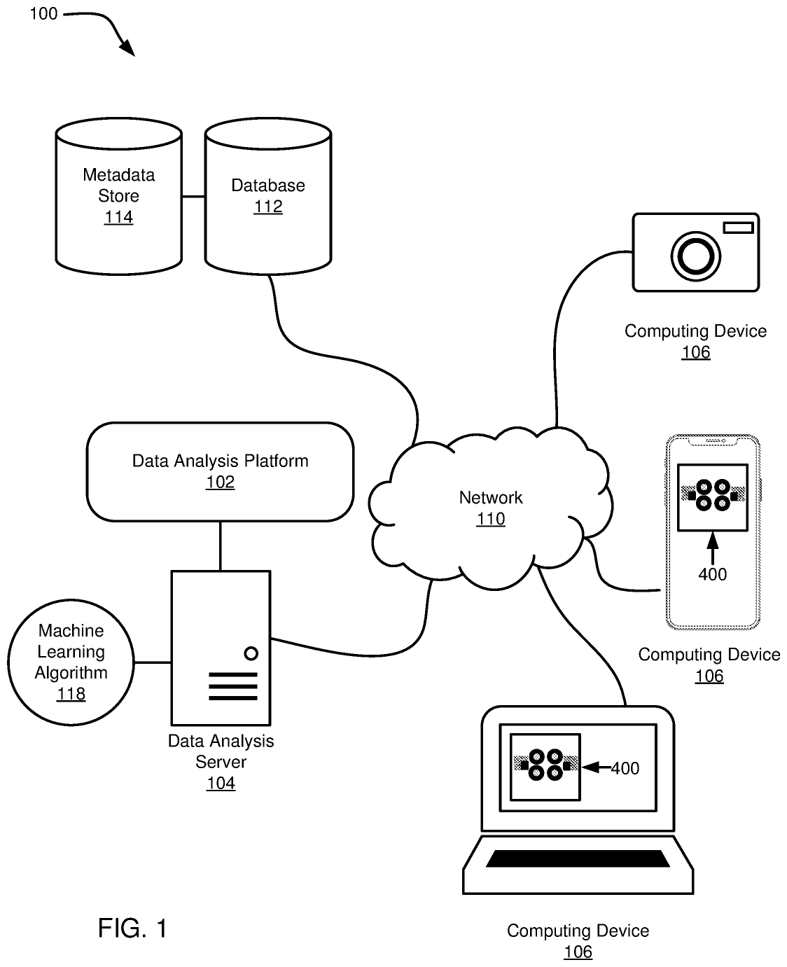

Referring now to the figures, FIG. 1 is a schematic diagram of a system 100 for remote data analysis. The system 100 may be implemented for point-of-care sample testing, and specifically for point-of-care blood sample and antibody testing. The system 100 includes a data analysis platform 102 operated by a data analysis server 104. The system 100 includes one or more computing devices 106 that receive and transmit information by way of the network 110. The system 100 includes a database 112 for storing ingested and partitioned data, training datasets, structured data, unstructured data, and so forth. The database 112 may further include or be associated with a metadata store 114. Each of the data analysis server 104, the database 112, and the computing devices 106 is in communication with a network 110 such as the Internet.

The data analysis platform 102 includes a user interface and logic for managing datasets, performing data analysis, and providing results. In a specific implementation, the data analysis platform 102 includes a user interface that enables healthcare providers, laboratories, and/or patients to submit test data for image analysis and access qualitative and quantitative results for the test data in near real-time. The data analysis platform 102 is executed by the data analysis server 104, which is in communication with other entities and databases by way of Application Program Interfaces (APIs), Secure File Transfer Protocols (SFTP), or other connections by way of the network 110.

The data analysis server 104 provides storage and processing resources to support the data analysis platform 102. The data analysis platform 102 is a system for executing image analysis algorithms on unstructured files, and specifically images of agglutination assay cards. The data analysis platform 102 is further configured to manage data pertaining to agglutination assays, including images of agglutination assay test cards, image analysis results output by a machine learning algorithm, lot numbers and serial numbers associated with agglutination assay test cards, patient data, and so forth.

The computing devices 106 include personal computing devices that can communicate with the data analysis server 104 by way of the network 110. The computing devices 106 may include, for example, mobile phones, laptops, personal computers, servers, server groups, tablets, image sensors, cameras, scanners, desktop computers, set-top boxes, gaming consoles, smart televisions, smart watches, fitness bands, optical head-mounted displays, virtual reality headsets, smart glasses, HDMI or other electronic display dongles, personal digital assistants, and/or another computing device comprising a processor (e.g., a central processing unit (CPU)), a processor core, image sensors, cameras, a field programmable gate array (FPGA), or other programmable logic, an application specific integrated circuit (ASIC), a controller, a microcontroller, and/or another semiconductor integrated circuit device, a volatile memory, and/or a non-volatile storage medium. The computing devices 106 may comprise processing resources for executing instructions stored in non-transitory computer readable storage media. These instructions may be incorporated in an application stored locally to the computing device 106, an application accessible on a web browser, and so forth. The application enables a user to access the user interface for the data analysis platform 102 to check submissions, upload files, verify whether files are accurately uploaded, receive feedback from the machine learning algorithm 118, and so forth.

The computing devices 106 transmit images to the data analysis server 104 such that the machine learning algorithm 118 can perform image analysis on the uploaded images. The computing devices 106 may include an integrated image sensor or may be in communication with an image sensor. The computing devices 106 may include memory for storing images captured by another device, and then transmit those images to the data analysis server 104. In some implementations, the computing devices 106 include a smart phone or tablet with an integrated image sensor.

In an implementation, the data analysis platform 102 is accessible on a mobile phone application. The mobile phone application uses the camera and networking capabilities of the mobile phone to capture test card images and upload those images to the data analysis server 104 and machine learning algorithm 118 for analysis. In an implementation, two QR codes, placed on the opposite sides of the test card, are used to identify manufacturing data about the test card and provide a unique ID for each test card. The mobile phone application also uses the QR codes to ensure the image is captured when the orientation and distance to the card are correct and the image is in focus. To ensure that the picture is taken at the correct time, the mobile phone application scans the QR codes and initiates a countdown timer that notifies a technician when the picture should be taken to produce a valid result. The countdown timer may be optimized to ensure that an agglutination reaction has had sufficient time to develop into a visually discernable agglutination condition. Additionally, the countdown timer may ensure the image is captured prior to the agglutination reaction becoming invalid, for example, due to excess drying time such that the agglutination condition can no longer be discerned by a human or the machine learning algorithm 118. The mobile phone application may be used in conjunction with an LED illumination box to maximize the consistency of the test card image captures. The test results may then be interpreted by the machine learning algorithm 118 and sent directly back to the mobile phone application for presentation at the point of care.

The system 100 enables users to access an account associated with the data analysis platform 102 by way of a computing device 106. The user may be assigned a security role and location access to as many, or few, entities as is required by the user's position. Security roles restrict what information and/or functionality the user can access. The data analysis platform 102 may be accessible on a mobile phone application. The mobile phone application uses the camera and networking capabilities of the mobile phone to capture images and upload those images to the data analysis server 104 and machine learning algorithm 118 for analysis.

The database 112 is a repository of information, datasets, images, structured data, unstructured data, and training datasets for the machine learning algorithm 118. The data analysis server 104 may access the database 112 by way of an Application Program Interface (API) over the network 110 connection. The API allows the data analysis server 104 to receive automatic updates from the database 112 as needed. In an embodiment, the database 112 is integrated on the data analysis server 104 and is not independent of the storage and processing resources dedicated to the data analysis server 104.

Data stored in the remote or cloud storage, such as the database 112, may include data, including images and related data, from many different entities, patients, laboratories, healthcare providers, locations, or the like. The stored data may be accessible to a classification system that includes a classification model, neural network, or other machine learning algorithm.

The database 112 may include a plurality of database instances, wherein each database instance is assigned to a different client account (e.g., a different laboratory or healthcare provider). In an implementation, the data analysis platform 102 is implemented for ingesting health test data for analysis, and in this case, the database 112 may include an independent database instance for each entity using the data analysis platform 102. In an alternative embodiment, the data for each entity is stored in the same physical memory location, and permissions for reading or writing the data is controlled by way of permissions managed by the data analysis platform 102.

The database 112 may be structured as a directed graph file system (which may be referred to as a semantic file system). The directed graph file system structures data according to semantics and intent, rather than location. The directed graph file system allows data to be addressed by content (associative access).

The database 112 may be structured as a relational database. In a relational database, files and data are stored with predefined relationships to one another. The files and data are organized as a set of tables with columns and rows, and tables are used to hold information about the objects to be represented in the database 112.

The machine learning algorithm 118 comprises storage and processing resources for executing an artificial intelligence algorithm. The machine learning algorithm 118 may be executed by a neural network and may specifically be executed by a deep learning convolutional neural network (CNN). The convolutional neural network is based on the shared weight architecture of convolution kernel or filters that slide along input features and provide translation equivalent responses known as feature maps. The machine learning algorithm 118 may include one or more independent neural networks trained to implement different machine learning processes.

In some implementations, the machine learning algorithm 118 is trained to classify the agglutination of a blood spot on a linear scale. This linear quantification may be used to determine whether the blood spot is agglutinated or non-agglutinated and may further be used to predict whether the blood spot has spontaneously agglutinated due to a patient's personal physiological characteristics, or whether the blood spot agglutinated due to reagents applied to the blood sample. The linear scale may include a scale from 0 to 10 or may include any suitable scale depending on the implementation. In an example implementation, the agglutination scale ranges from 0 to 10, wherein a non-agglutinated sample is scored by the machine learning algorithm 118 as having a score of 0 or 1. An agglutinated sample may be scored by the machine learning algorithm 118 as having a score of 7, 8, 9, or 10. The machine learning algorithm 118 may score semi-agglutinated samples as having a score of 2, 3, 4, 5, or 6, and these samples may be retested, evaluated visually by a human, or determined to indicate the patient's sample spontaneously agglutinated. The linear scale may include delineated thresholds, such as 1, 2, 3, 4, 5, 6, and so forth as described herein. The linear scale may further include graduated thresholds such that the agglutination may be quantified with intermediate numbers such as 1.5, 2.3, 8.7, and so forth.

It should be appreciated that the machine learning algorithm 118 may be trained to quantify the agglutination on any suitable scale, including the linear scale specifically described herein, along with logarithmic scales, exponential scales, and other non-linear scales. The machine learning algorithm 118 calibrates the quantitative scale based on one or more control samples. The control samples may include a negative control sample, positive control sample, and/or midrange calibrator sample as described herein. The machine learning algorithm 118 shifts the quantitative scale based on the visual agglutination of the control samples to account for the patient's natural “background agglutination,” which is a result of the patient's physiological propensity for blood agglutination. The machine learning algorithm 118 classifies the agglutination of the patient's one or more test samples by plotting the test sample on the calibrated quantitative scale.

The machine learning algorithm 118 is further trained to determine whether a blood sample is agglutinated or non-agglutinated without assigning a linear classification number to the sample. The machine learning algorithm 118 is trained on a dataset comprising agglutinated samples and non-agglutinated samples as discussed further herein. The machine learning algorithm 118 is trained on datasets comprising numerous categories of patients, including images of treated blood spots from combinations of patients who have been vaccinated against a virus, patients who have not been vaccinated against the virus, patients who are currently infected with the virus, patients who have never been infected with the virus, and patients who were previously infected by the virus but have since recovered. The machine learning algorithm 118 is trained on this dataset to assess a new sample image of a blood spot to determine whether the patient has antibodies for the virus present in the patient's bloodstream. These antibodies can indicate whether the user has been vaccinated, has previously been infected with the virus, is currently infected with the virus, and so forth.

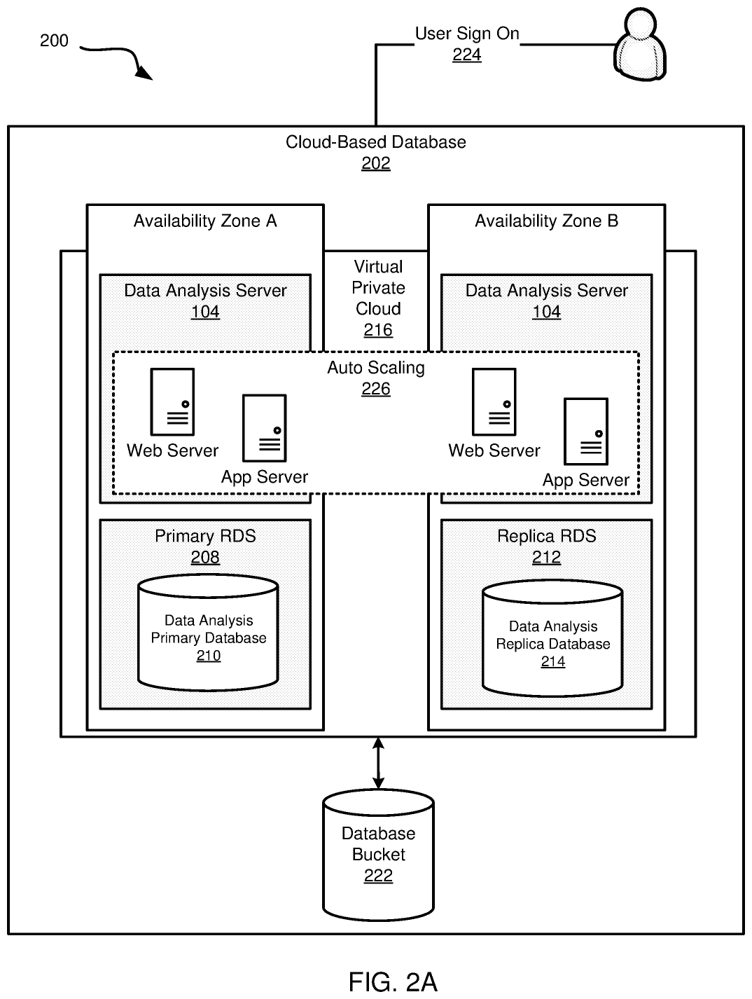

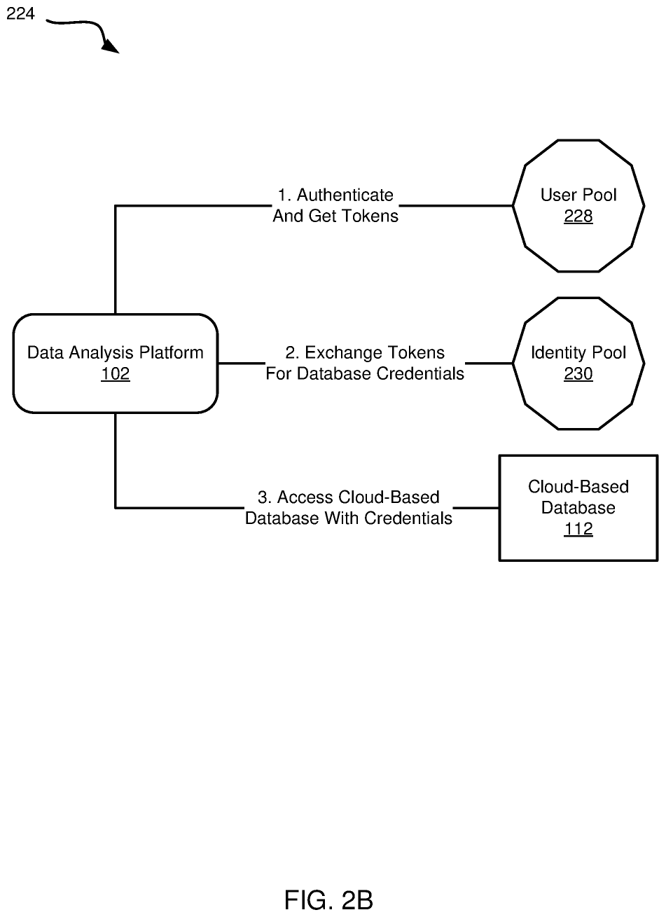

FIG. 2A is a schematic block diagram of a system 200 for data management and security. The system 200 illustrated in FIG. 2A is implemented in conjunction with the system 100 illustrated in FIG. 1. The system 200 includes the cloud-based database 112 supporting the data analysis server 104. The cloud-based database 112 includes an Availability Zone A and an Availability Zone B. The Availability Zone A includes a first instance of the data analysis server 104 and the Availability Zone B includes another instance of the data analysis server 104. Each of the instances of the data analysis server 104 includes a web server and an app server, and the cloud-based database 112 auto-scales the processing and storage resources between the web servers and app servers for the Availability Zone A and the Availability Zone B. The Availability Zone A includes a primary relational database service (RDS) 208 and the Availability Zone B includes a replica relational database service 212. The data analysis primary database 210 is stored on the primary relational database service 208 and the data analysis replica database 214 is stored on the replica relational database service 212. The virtual private cloud 216 of the cloud-based database 112 communicates with outside parties by way of Application Program Interfaces and Secure File Transfer Protocol (SFTP) messaging. The cloud-based database 112 includes a database bucket 222 for storing information associated with the data analysis platform 102. Users interacting the data analysis platform 102 can sign on 224 to the service by communicating with the cloud-based database 112.

The cloud-based database 112 includes processing and storage resources in communication with the network 110. The cloud-based database 112 includes a resource manager for managing the usage of processing and storage resources. The resource manager of the cloud-based database 112 performs auto scaling 226 load balancing to ensure adequate processing and storage resources are available on demand based on real-time usage.

The availability zones represent discrete datacenters with redundant power, networking, and connectivity for supporting the data analysis server 104. The availability zones enable the ability to operate production applications and databases in a more highly available, fault tolerant, and scalable way than would be possible with a single datacenter. The Availability Zone A and Availability Zone B are interconnected with high-bandwidth, low-latency networking, over fully redundant, dedicated metro fiber providing high-throughput, low-latency networking between the availability zones. All traffic between the availability zones may be encrypted. The network performance of the availability zones is sufficient to accomplish synchronous replication between the availability zones. Applications, modules, components, and processing methods can be partitioned between the availability zones of the cloud-based database 112. When applications are partitioned across the availability zones, the data analysis server 104 operates with increased protection and isolation from outages that may be caused by a low in power, hardware issues, software issues, and so forth. The availability zones are physically separated by a meaningful geographic distance to ensure the hardware supporting the availability zones will not be impacted by the same outside forces, such as power outages, natural disasters, and so forth.

The virtual private cloud 216 is an on-demand configurable pool of shared resources allocated within the cloud-based database 112. The virtual private cloud 216 provides isolation between different users communicating with the cloud-based database 112, e.g., different facilities, user accounts, and clients in communication with the data analysis platform 102. The isolation between one virtual private cloud 216 user and all other users of the same cloud is achieved through allocation of a private IP subnet and a virtual communication construction such as a VLAN or a set of encrypted communication channels per user. The virtual private cloud 216 provides isolation between users within the cloud-based database 112 and is accompanied with a VPN function allocated per-user within the virtual private cloud 216. This secures the remote access to the data analysis platform 102 by way of authentication and encryption. The data analysis platform 102 is then essential run on a “virtually private” cloud, even if the processing and storage resources are provided by a third-party cloud-based database service, such as Amazon Web Services®.

The auto-scaling 226 is performed by a resource manager of the cloud-based database 112. The resource manager distributes workload between the web servers and the app servers of the various availability zones of the cloud-based database 112. In some cases, one client of the data analysis platform 102 may consume a large quantity of storage resources and processing resources at a certain time, and the resource manager will allocate different web servers and app servers across the availability zones to ensure the client receives an adequate quantity of storage and processing resources. The auto-scaling 226 is performed in real-time to meet the needs of the data analysis platform 102.

The primary and secondary relational database services 208, 212 provide a means to access, replicate, query, and write to the data analysis database instances 210, 214. The data analysis primary database 210 may include a copy of data associated with the data analysis platform 102, such as user login information, user preference data, pharmaceutical inventory data, past purchasing data, current pricing data, pharmaceutical catalogue data, and so forth. The data analysis replica database 214 may include a replica copy of all or some of the data stored on the data analysis primary database 210. The replicated databases provide fault-tolerance and protect the data analysis platform 102 form becoming inoperative during a power outage, hardware outage, or natural disaster.

The database bucket 222 provides object storage through a web service interface. The database bucket 222 uses scalable storage infrastructure that can be employed to store any type of object. The database bucket 222 may store applications, software code, backup and recovery, disaster recovery, data archives, data lakes for analytics, and hybrid cloud storage to support the data analysis platform 102.

FIG. 2B is a schematic block diagram of a system and process flow for accessing the cloud-based database 112 described in FIG. 2A. The data analysis platform 102 first authenticates and retrieves tokens from a user pool 228. The data analysis platform 102 then exchanges tokens for database credentials with the identity pool 230. The data analysis platform 102 is then granted access to the could-based database 112 based upon the credentials.

The user pool 228 is a user directory associated with the cloud-based database 112. With the user pool 228, users can sign into the data analysis platform 102 through a mobile application, computer-based application, web-based user interface, third-party identity provider, and so forth. Whether users sign in directly or through a third party, all members of the user pool 228 have a director profile that can be accessed. The user pool 228 enables sign-up and sign-in services for the data analysis platform 102 and further enables social sign-in with outside services, including outside social media networks. The user pool 228 stores a directory, and this directory may be managed, and permissions may be assigned to users within the director.

The identity pool 230 creates temporary credentials to access the cloud-based database 112. The identity pool 230 supports anonymous guest users and social sign-in through outside parties, including third-party social media network.

The system 200 authenticates users by leveraging the user pool 228. After a successful sign-in through the user pool 228, the data analysis platform 102 creates user pool groups to manage permissions and to represent different types of users. The data analysis platform 102 creates user groups defined by a type of data permission for that group.

The data analysis platform 102 may access the cloud-based database 112 through an Application Program Interface (API) Gateway. The API Gateway validates the tokens from a successful user pool 228 authentication and uses those tokens to grant users access to the resources within the data analysis platform 102 and the cloud-based database 112. The data analysis platform 102 leverages the user groups defined within the user pool 228 to control permissions with the API Gateway by mapping group membership to roles within the user pool 228. The user groups that a user is a member of are included in the identification token provided by a user pool 228 when the user signs into the data analysis platform 102. The data analysis platform 102 submits the user pool tokens with a request to the API Gateway for verification by an authorizer for the cloud-based database 112.

In an embodiment, the data analysis platform 102 implements role-based access control. The identity pools 230 assign authenticated users a set of temporary, limited-privilege credentials to access the resources in the cloud-based database 112. The permissions for each user are controlled through roles created within the data analysis platform 102. The data analysis platform 102 defines rules to choose the role for each user based on claims in the user's identification token. The rules enable the data analysis platform 102 to map claims from an identity provider token to a role. Each rule specifies a token claim (such as a user attribute in the identification token from the user pool 228), match type, a value, and a role. The match type can be Equals, NotEqual, StartsWith, or Contains. If a user has a matching value for the claim, the user can assume that role when the user gets credentials. For example, the data analysis platform 102 may create a rule that assigns a specific role for the users with a custom:dept custom attribute value of Sales.

Rules are evaluated in order, and the role for the first matching rule is used, unless a custom role is specified to override the order. The data analysis platform 102 may set multiple rules for an authentication provider in the identity pool 230. Rules are applied in order. The order of the rules may be altered. The first matching rule takes precedence. If the match type is NotEqual, and the claim does not exist, then the rule is not evaluated. If no rules match, the role resolution setting is applied to either use the default authenticated role or to deny. The data analysis platform 102 specifies a role within the API connection to the cloud-based database 112 to be assigned when no rules match in the ambiguous role resolution process. For each user pool 228 or other authentication provider configured for an identity pool 230, the data analysis platform 102 may assign numerous rules.

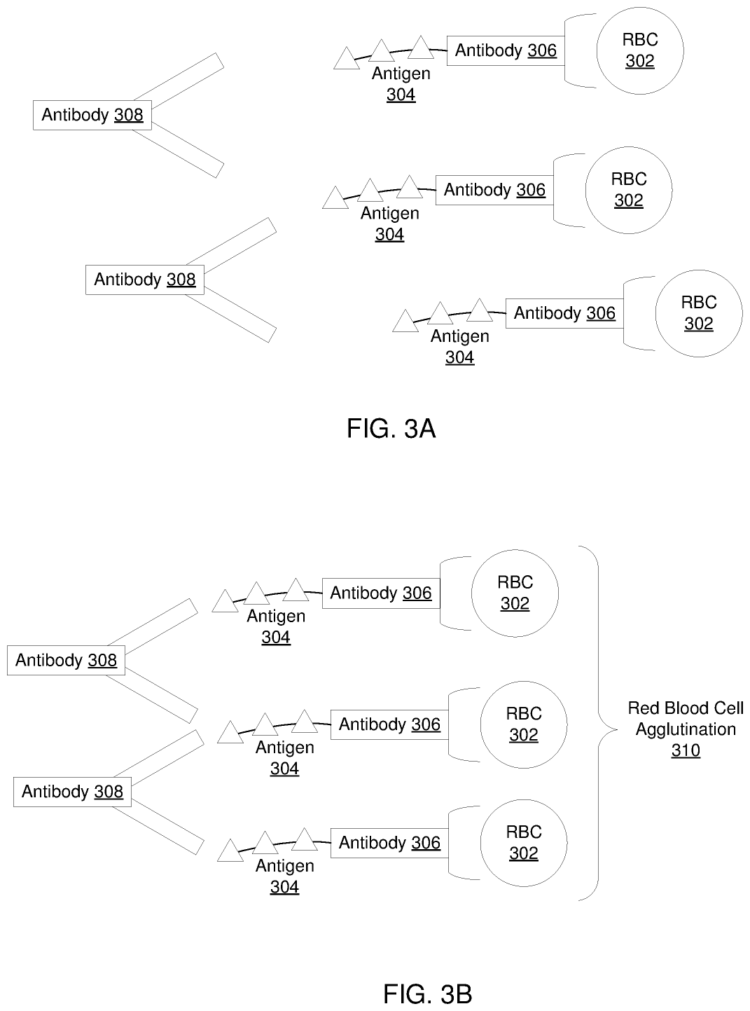

FIGS. 3A and 3B are schematic illustrations of blood agglutination to provide further background and understanding for the systems, methods, and devices described herein. Hemagglutination occurs when an antigen is mixed with its corresponding antibody in the blood.

Blood includes at least the red blood cells (RBCs) 302, antigens 304, and antibodies 306, 308 illustrated in FIGS. 3A-3B. The antigens 304 may include one or more of heteroantigens (i.e., foreign agents) and autoantigens (i.e., self-antigens). Heteroantigens originate from outside the body and may include, for example, parts of or substances produced by viruses or microorganisms such as bacteria and protozoa, as well as substances in venom, certain proteins in foods, and components of serum and red blood cells from other individuals. Autoantigens originate with the body and may trigger an immune response in persons with an autoimmune disorder. The antigen 304 may be coupled to antibodies 306 that bind to red blood cells 302 as shown in FIGS. 3A-3B.

FIG. 3A illustrates an instance where agglutination has not occurred because the antibodies 308 within the blood do not correspond to the antigens 304. FIG. 3B illustrates an instance where agglutination is occurring because the antibodies 308 correspond with and bind to the antigens 304. This causes the blood to clump together, or “agglutinate.”

One antigen specifically described herein is the SARS-CoV-2 virus and its associated spike proteins. Described herein are diagnostic tests and image analysis for detecting whether a patient has antibodies against the SARS-CoV-2 virus. The diagnostic tests described herein include hemagglutination-based tests based on bispecific proteins that contain a dromedary-derived antibody (nanobody) that binds red blood cells and a SARS-CoV-2-derived antigen on the receptor-binding domain of the spike protein or the nucleocapsid protein.

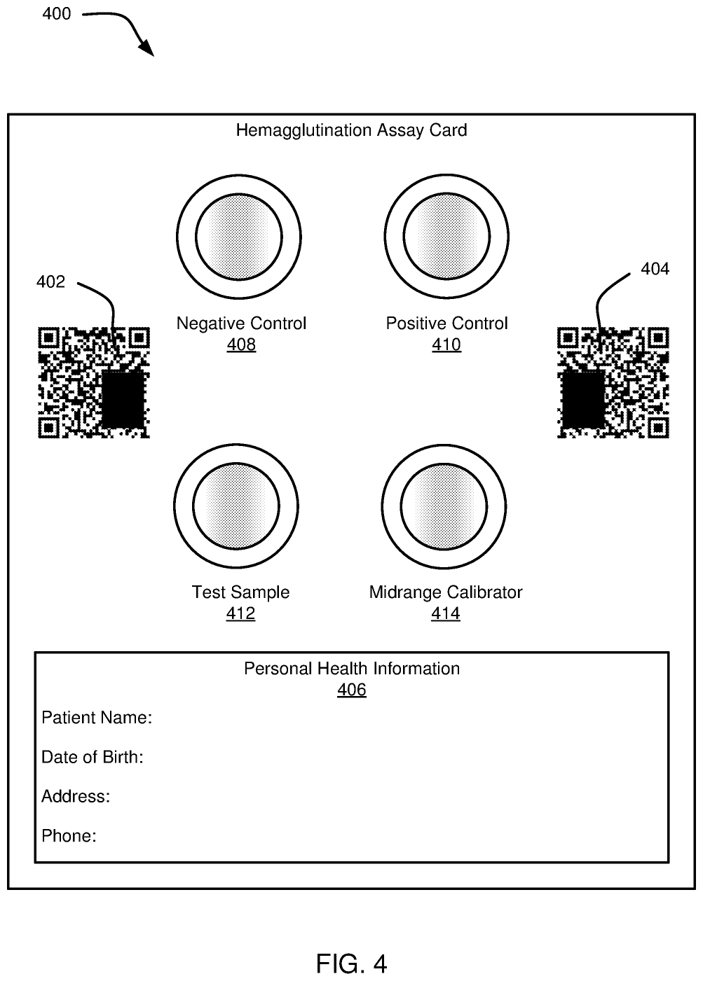

FIG. 4 is a schematic illustration of an example test card 400. The test card 400 is implemented with the systems and methods described herein for qualitatively and quantitatively assessing the agglutination of blood samples. In an implementation, a user captures an image of the test card 400 and uploads that image to the data analysis platform 102 for analysis. The machine learning algorithm 118 performs image analysis on the image of the test card and outputs a result. This may be performed at point-of-care shortly after a patient provides the necessary blood samples.

The example test card 400 illustrated in FIG. 4 is a hemagglutination assay card comprising four sample deposit regions configured for receiving a blood sample. The sample deposit regions may be configured for receiving a small blood sample that may be retrieved with a fingerstick sampling or other means. It should be appreciated that the blood sample may be retrieved by one or more of a fingerstick sampling, venipuncture sampling, or arterial sampling, depending on which is most convenient or desirable in the use-case. The test card 400 includes one or more of a negative control sample 408, a positive control sample 410, a test sample 412, and a midrange calibrator 414. It should be appreciated that the test card 400 may have varying numbers of samples depending on the application. In some implementations, the test card 400 may have the negative control sample 408, the positive control sample 410, the midrange calibrator 414, and any number of test samples 412. The test card 400 may include only the negative control sample 408 and the positive control sample 410 for calibration purposes and emit the midrange calibrator 414. The test card 400 may include one or more regions for depositing a test sample 412.

The testing region for the negative control sample 408 comprises a reagent deposited on the test card 400. This reagent does not induce any agglutination of the patient's blood sample and may prevent agglutination of the patient's blood sample. The negative control reagent may include a protein that lacks an antigen associated with the test samples 412.

The testing region for the positive control sample 410 includes a reagent deposited on the test card 400 that is configured to induce agglutination of a blood sample. The positive reagent includes, for example, an antibody, combination of antibodies, or other affinity reagent that leads to high degrees of agglutination in a blood sample, such that the blood sample is visibly clumped together. The positive control reagent may specifically include a nanobody that associates or binds to red blood cells and antibodies directed against the nanobody. This mimics agglutination induced by physiological antibodies contained in patient serum. In this case, the positive control reagent acts as a surrogate for a physiological blood response to the antigen and will induce agglutination of the positive control sample 410.

The testing regions for the one or more test samples 412 comprise a testing reagent deposited on the test card 400. The testing reagent may include a bi-functional recombinant protein that binds red blood cells and contains an identified antigen. The identified antigen will vary depending on the implementation and may include any substance capable of stimulating an immune response by the patient's body. The antigen may specifically include a foreign antigen, such as parts or substances produced by viruses, bacteria, or protozoa, as well as substances in venom, certain proteins in foods, and components of serum and red blood cells from other individuals. The antigen may be an autoantigen, such as proteins or other substances created by a patient with an autoimmune disorder.

The testing region for the midrange calibrator 414 includes a reagent that induces agglutination and further includes a “standard” for an identified antibody (i.e., the antibody being tested for with the test card 400). The standard for the identified antibody includes a standardized quantity of the identified antibody and may be acquired from a centralized agency such as the World Health Organization® or Centers for Disease Control and Prevention®. When the blood sample is deposited on the midrange calibrator 414 testing region, the blood sample will combine with the standardized antibody within the midrange calibrator 414. This provides an indication of how the blood sample will agglutinate when the blood sample is known to include at least some quantity of the standardized antibody. The machine learning algorithm 118 utilizes the midrange calibrator 114 to adjust the scale according to the personal physiological characteristics of the patient providing the blood samples. The machine learning algorithm 118 thereby adjusts the qualitative and quantitative assessments of the patient's blood agglutination based at least in part on the degrees of agglutination seen in the negative control 408, positive control, and/or midrange calibrator 414.

In an implementation, the machine learning algorithm 118 calibrates a quantitative scale based on visual agglutination of the negative control 408, the positive control 410, and the midrange calibrator 414. Depending on the implementation and the antigen/antibody being assessed, one or more of the negative control 408, the positive control 410, or the midrange calibrator 414 may be eliminated from the analysis. The machine learning algorithm 118 calibrates the quantitative scale to account for the patient's natural “background agglutination,” which is a result of the patient's unique physiological makeup. The patient's background agglutination is antigen-agnostic and represents the natural propensity for the patient's blood to agglutinate. The quantitative scale is calibrated for each test card 400 to ensure the results of the test sample 412 agglutination are normalized across a population to account for each patient's natural background agglutination.

The quantitative scale may include a linear scale or non-linear scale. The quantitative scale may specifically include a logarithmic scale. It should be appreciated that the type of scale may depend on the antigen/antibody being evaluated with the test card 400. The quantitative scale enables the machine learning algorithm 118 and/or a human user to quantitatively assess the patient's hemagglutination based on the blood samples deposited on the test card 400. This quantitative assessment enables the machine learning algorithm 118 and/or a human user to more accurately predict whether the patient has been exposed to the identified antigen being evaluated for or currently possess antibodies for the identified antigen. Additionally, the quantitative scale is used to qualitatively assess agglutination of the test sample 412 by classifying the agglutination as “low,” “moderate,” “high,” and so forth depending on where the test sample 412 falls on the quantitative scale. Additionally, the qualitative assessment may include a prediction of whether the patient has been exposed to the antigen, vaccinated against the antigen, and/or currently possesses antibodies against the antigen.

The test card 400 includes a first unique code 402 and a second unique code 404. The unique codes 402, 404 may include a Quick Response (QR) code as shown in FIG. 4. The unique codes 402, 404 are collectively used to identify manufacturing data for the test card 400 and provide a unique identifier for each test card, which may be matched with a patient and a certain blood sample retrieved from the patient. The serial number and/or lot number for the test card 400 may be stored in one or more of the unique codes 402, 404.

One or more of the unique codes 402, 404 provide instructions to a computing device 106 that is capturing an image of the test card 400. The computing device 106 is instructed to capture an image of the test card 400 that comprises one or more of the correct orientation, framing, exposure, focus, white balance, and so forth. In an implementation, one or more of the unique codes 402, 404 provides instructions to the computing device 106 to initiate a countdown timer when the image of the test card 400 is in focus, and then cause the computing device 106 to automatically capture an image of the test card 400 or provide a notification that a user should capture an image of the test card 400. Thus, the unique codes 402, 404 provide instructions to the computing device 106 to ensure that the image of the test card 400 is sufficiently framed, exposed, and in-focus to ensure the machine learning algorithm 118 can accurately analyze the test card 400.

In an implementation, the orientation and/or positioning of the unique codes 402, 404 is optimized to ensure that the computing device 106 captures a valid image of the test card 400. The unique codes 402, 404 may be positioned to ensure the image sensor in communication with the computing device 106 is properly positioned over the test card 400 to capture a valid image of the test card 400 that can be processed by the machine learning algorithm 118. The unique codes 402, 404 are printed on the test card 400 such that they are positioned at the same focal distance along a Z-axis (from the surface of the test card 400 to an image sensor positioned above the test card 400) as the blood spot samples 408-414.

The test card 400 includes a section for personal health information 406, such as the patient's name, date of birth, health history, blood type, vaccination history, antigen exposure history, contact information, and so forth. The personal health information 406 may be manually filled out by a user or may be printed on to the test card 400. The personal health information 406 may be “scrubbed” such that it includes only anonymous codes or other identifiers for the patient that cannot be readily understood by another person.

The one or more unique codes 402, 404 may provide instructions to the computing device 106 to frame an image of the test card 400 such that the personal health information 406 is not captured in the image. Additionally, these instructions may be a component of a software application installed on the computing device 106 that is configured to facilitate image capture of the test cards 400. In an implementation, the computing device 106 captures a first image that includes the personal health information 406 and captures a second image that does not include the personal health information 406. Additionally, the computing device 106 may be configured to crop a test image to remove the box for the personal health information 406 such that only an image of the test samples 408-414 and the unique codes 402, 404 remains. The computing device 106 ensures that the personal health information 406 is only stored locally on the computing device 106 and is never uploaded to the data analysis platform 102. This protects the personal health information 406 from being intercepted or retrieved if the database 112 and/or data analysis server 104 is hacked.

Each of the blood samples 408-414 are retrieved from a single patient at one time. The blood samples 408-414 may specifically be retrieved by way of a fingerstick sampling on the patient, and then one or more drops of blood may be dropped on each testing region. It should be appreciated that the blood samples may be retrieved by any suitable means, including a venous blood draw.

The first unique code 402 and the second unique code 404 can be scanned for bookkeeping purposes in organizing data associated with the test card 400. The unique codes 402, 404 may provide information regarding, for example, the type of assay, the type of reagent used to treat the blood sample, identifying information for the patient, identifying information for the administering facility and/or professional, manufacturing data for the test card and/or the reagent used for the test, instructions for how the image should be captured, and so forth.

In one implementation, a unique code 402, 404 provides instructions to a computing device 106 on how to capture an image of the test card 400. These instructions may include, for example, instructions regarding lighting, orientation, distance from card, focus, orientation, and so forth. The unique codes 402, 404 may further include data pertaining to the lot number or serial number of the test card 400. The unique codes 402, 404 may include an expiration date for the test card. In some instances, the unique codes 402, 404 may instruct a computing device to “reject” a test card or refuse to capture an image of the test card 400 if the test card 400 is expired.

The unique codes 402, 404 may further include orientation indications, for example, the first unique code 402 on the left-side of the test card 400 may include data indicating it is positioned to the left of the test samples; similarly, the second unique code 404 on the right side of the test card 400 may include data indicating it is positioned to the right of the test samples. It should be appreciated that the layout of the test card 400 may be altered, and the orientation embedded within the unique codes 402, 404 will be dependent on the orientation and layout of the test card 400.

In an implementation, one or more of the unique codes 402, 404 provides instructions to the computing device 106 to ensure that the personal health information 406 is not captured in the image that is uploaded to the data analysis platform 102. The unique codes 402, 404 are serialized to ensure that patient health information is not associated with the image, and thus is not transmitted to a remote server for image analysis. When the machine learning algorithm 118 returns the results for the test card, these results are then recombined with the unique serial numbers associated with the unique codes 402,404. The computing device then relinks the patient health information to the results for final report generation.

In an implementation, the test card 400 is equipped with recombinant proteins for identify antibodies for the SARS-CoV-2 virus. In this implementation, the test card 400 may include a GPA-directed nanobody IH4. The IH4 nanobody includes two viral antigens, including the receptor-binding domain of the Spike protein (Spike-RBC) of SARS-CoV-2 (NanoSpike) and the Nucleocapsid (NanoNuc). Antibodies against the Spike-RBC correlate with neutralizing activity against the virus in both adults and children. In a range of 3-30 μg this protein allows assessment of successful vaccination. Antibodies against the Nucleocapsid are also found in most SARS-CoV-2-infected individuals, and antibody titers are particularly high in severe cases. Because most SARS-CoV-2 vaccines are based on the Spike protein, Nucleocapsid-directed AB can be used to identify AB obtained during natural infection. The spike protein is physiologically targeted to the secretory pathway, which is likely required for proper protein folding and accompanied by glycosylation, which in turn may be relevant for the AB response and test specificity.

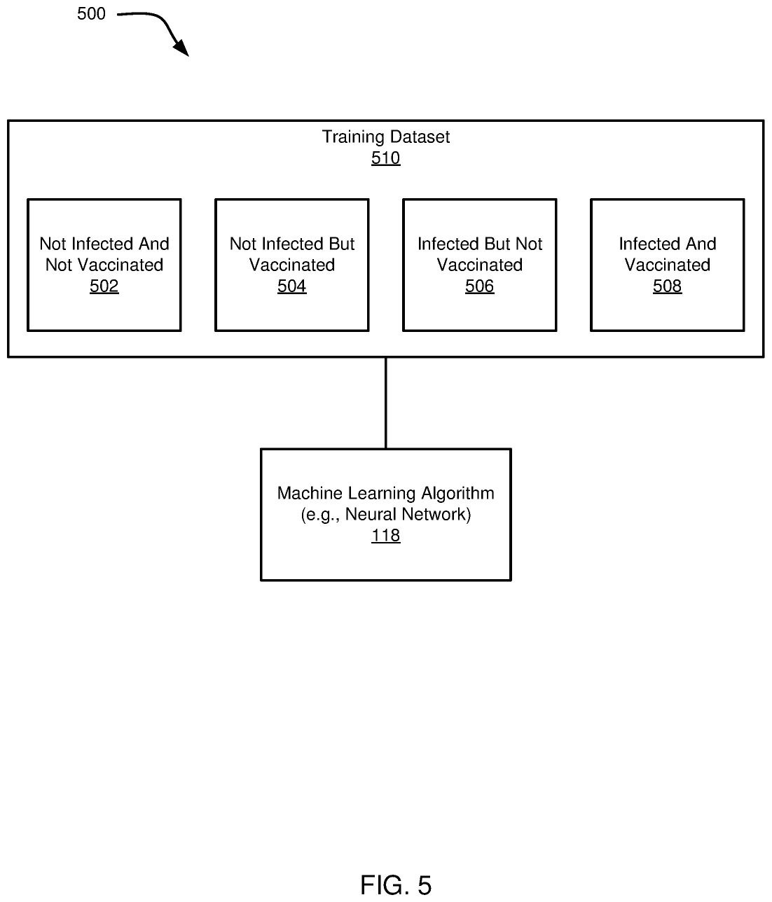

FIG. 5 is a schematic diagram of a training methodology 500 for training the machine learning algorithm 118 to identify positive and negative agglutination both qualitatively and quantitatively. The training methodology 500 includes providing a training dataset 510 to the machine learning algorithm 118.

The machine learning algorithm 118 is trained to classify the agglutination of one or more of a patient's blood test samples 412 based at least in part on the agglutination of the patient's blood in the negative control sample 408 and the positive control sample 410. The machine learning algorithm 118 calibrates a unique agglutination scale for each test card 400 based on the agglutination of the negative control sample 408 and the positive control sample 410. The machine learning algorithm 118 must recalibrate for each test card 400 because blood samples from different patients will naturally have different agglutination characteristics.

The training dataset 510 includes a plurality of training images. The training images include depictions of sample test cards 400 for one type of agglutination assay. The training dataset 510 described in FIG. 5 is specifically implemented to train the machine learning algorithm 118 to qualitatively and quantitatively determine whether the patient currently has antibodies for a certain antigen.

The training dataset 510 includes a plurality of training images depicting a test card 400 for subjects that have not been infected with the antigen and have not been vaccinated against the antigen (see 502), and therefore do not presently have antibodies for the antigen. The training dataset 510 includes a plurality of training images depicting a test card 400 for subjects that have not been infected with the antigen but have been vaccinated against the antigen (see 504). The training dataset 510 includes a plurality of training images depicting a test card 400 for subjects that have been infected with the antigen but have not been vaccinated against the antigen (see 506). The training dataset 510 includes a plurality of training images depicting a test card 400 for subjects that have been infected with the antigen and have also been vaccinated against the antigen (see 508). Each of the dataset categories 502-508 may be further partitioned into sub-categories. For example, the dataset categories may be partitioned based on the manufacturer of the vaccine, when the patient received the vaccine, how many doses of the vaccine the patient received, and so forth. Further for example, the dataset categories may be partitioned based on when the patient was infected with the antigen, how many times the patient was infected with the antigen, whether the patient continues to test positive for being infected with the antigen, which variation of the antigen the patient was infected with, and so forth.

The machine learning algorithm 118 is trained to assess the agglutination assays from each of the training dataset samples 502-508 and identify differences and similarities between those images. The machine learning algorithm 118 thereby learns to classify whether a patient has likely been vaccinated or infected with a certain antigen based on the patient's agglutination assay on the test card 400. The machine learning algorithm 118 may provide a quantitative likelihood of whether the patient has been vaccinated against the antigen or infected with the antigen based on the visual characteristics of the patient's agglutination assay on the test card 400. Additionally, the machine learning algorithm 118 outputs a qualitative and/or quantitative indication of whether the patient currently has a non-detectable, low, moderate, or high quantity of antibodies against the antigen.

In an implementation, the training dataset 510 is specifically applied to identifying SARS-CoV-2 antibodies. In this implementation, the training dataset 510 includes four data categories. The training dataset 510 includes images of test card 400 blood samples from patients who have not been infected by SARS-CoV-2 and have not been vaccinated against SARS-CoV-2 (see 502). The training dataset 510 includes images of test card 400 blood samples from patients who have not been infected by SARS-CoV-2 but have been vaccinated against SARS-CoV-2 (see 504). The training dataset 510 may further be portioned based on the manufacturer of the vaccine against SARS-CoV-2, when the patient was vaccinated, how many doses of the vaccine the patient received, and so forth. The training dataset 510 includes images of test card 400 blood samples from patients who have been infected with SARS-CoV-2 and have not been vaccinated against SARS-CoV-2 (see 506). The training dataset 510 includes images of test card 400 blood samples from patients who have been infected with SARS-CoV-2 and have been vaccinated against SARS-CoV-2. In other implementations, the training dataset 510 includes categories for patients who have been infected or interacted with any antigen, such as bacteria, viruses, foreign bodies, and so forth. It should be appreciated that the hemagglutination test described herein detects antibodies present in a patient's blood, and the patient's body may create those antibodies in response to any antigen, and not necessarily in response to a virus.

The machine learning algorithm 118 may be based on YOLOv3 with anchor boxes set to 500 pixels square to identify agglutinated and non-agglutinated blood spots. Potential training labels are statistically divided into Training and Validation sets in an approximately 9:1 ratio for each class. Training labels are oversampled such that every dataset category includes an equal number of presentations to the model in training. The resulting set of training images is presented to the machine learning algorithm 118 in each epoch, and the validation images are used to measure training progress after each epoch. Training images are randomly flipped and rotated at 90-degree intervals, and image hue and saturation are randomly augmented by as much as 10 percent. The machine learning algorithm 118 is trained using stochastic gradient descent with backpropagation using a batch size of 12 and starting with a learning rate of 1e-4 which decreased over 7 epochs to 1e-5. Because training and validation images are selected from a common set of image captures, a holdout set composed of distinct image captures that are withheld from the training process used to test the final trained machine learning algorithm 118 for generalized performance. The machine learning algorithm 118 predictions consist of object bounding box location and size, object type (i.e., agglutinated blood spot, non-agglutinated blood spot), and confidence value in range, [0, 1]. An arbitrary minimum-confidence threshold of 0.2 is used to filter out spurious predictions. On the holdout set, the machine learning algorithm 118 produces high-confidence spurious prediction.

Patient blood for generating the training dataset 510 may be gathered by fingerstick sampling in many applications. In parallel, venous blood samples may be obtained for approved tests to substantiate the results of the machine learning algorithm 118 image analysis. It should be appreciated that the blood sample may be collected by any suitable means, including a fingerstick sampling, venipuncture sampling, or arterial sampling. The complete set of the results is provided to the machine learning algorithm 118. Patient samples are analyzed except for samples where image quality obtained by the assay was insufficient or the confidence score was below the predetermined threshold. The machine learning algorithm 118 ranks assay tests with a positive score if analyte and positive controls were two or more bins above negative control. The machine learning algorithm 118 ranks assay tests with an equivocal score if analyte was only one bin above negative control. The machine learning algorithm 118 ranks assay tests with a negative score if analyte was in same bin or below the negative control.

The machine learning algorithm 118 can be trained with only blood spot images with and without apparent agglutination. However, this plus/minus interpretation does not allow differentiation of spontaneous agglutination (necessitating invalidation of some tests) and does not allow for quantification of antibody titers. In a proof-of-concept application, the machine learning algorithm 118 is trained with 10 bins representing arbitrary levels of increasing agglutination to generate a discontinuous standard curve ranging from non-detectable to maximal agglutination. Training the model on these bins resulted in a correlation between visual and computer-based image interpretation, achieving a test performance in a small clinical study that was comparable to standard EUA-approved tests. Importantly, the assay described herein enabled impartial test analysis, free of the subjectivity of individual investigators.

The analysis output of the machine learning algorithm 118 can be improved through the generation of a continuous standard curve (as opposed to the discontinuous bins). This may be accomplished through generation of a regression curve based on existing bin-labeled data or, alternatively, based on de-novo, pairwise-comparison of large numbers of spots with different levels of agglutination by human investigators to generate a more fine-grained agglutination ranking scale, followed by re-training of the machine learning algorithm 118. More precise methods improve overall quantification and allow for more accurate test interpretation, particularly for low-titer cases. Specifically, by broadening the spectrum of agglutination samples, including those with imperfections, such as air bubbles or sample preparation issues, the machine learning algorithm 118 will be empowered to ignore such artifacts or, if significant, to invalidate the test upon detecting them.

Group one patients (no infection, no vaccination) scored negative in processing tests of the machine learning algorithm 118, suggesting comparably high specificity. Likewise, patients who had been vaccinated (groups two and four) were identified coherently positive by all Spike tests, including the assay described herein. While group two patients (no infection, vaccination) were negative in the Nucleocapsid test, some patients of group four (infected, vaccinated) were Nucleocapsid-positive, as expected. The results of group three patients (infected, not vaccinated) were more heterogeneous due to variable, in part low antibody titers as apparent by low neural network training datasets or arbitrary units (ELISA, CLIA).

Historically, visual approaches to identifying hemagglutination focus on qualitative test interpretation, even though visual inspection provides a certain level of relative quantification because test and control samples are directly compared to each other. Such comparative analysis is particularly useful for samples showing minor, spontaneous agglutination, which is observed in rare conditions, such as immune-mediated hemolytic anemia. Computer-based interpretation using the algorithm described herein identifies control samples as agglutination-positive with the consequence to invalidate such tests.

The agglutination identification algorithm described herein implements a discrete or continuous quantification model. The agglutination identification algorithm is implemented as a generalization from two original labels, including, “Agglutination negative” and “Agglutination positive” to 10 new labels: “Agglutination 0” (no agglutination) through “Agglutination 9” (maximum agglutination). The samples may be divided into training and validation labels, followed by training of a deep learning object detection model to identify the location, type (i.e., Agglutination 0-9), and confidence for each blood spot in a manner like the previous binary classifier. After training, the model is tested on a holdout set containing samples that had not been used for training. On the expanded holdout test set, the maximum spurious prediction confidence was 0.41, and the minimum true positive blood spot prediction confidence was 0.53. Thus, for subsequent processing, predictions below a confidence threshold of 0.5 were considered spurious. Table 1 indicates results of an implementation of the deep learning model described herein.

TABLE 1

| | Training | Validation | Holdout Test |
|---|---|---|---|
| Agglutination 0 | 1050 | 117 | 102 |
| Agglutination 1 | 153 | 18 | 19 |
| Agglutination 2 | 109 | 13 | 12 |
| Agglutination 3 | 171 | 20 | 15 |
| Agglutination 4 | 75 | 9 | 14 |
| Agglutination 5 | 67 | 8 | 13 |
| Agglutination 6 | 184 | 21 | 19 |
| Agglutination 7 | 204 | 23 | 18 |
| Agglutination 8 | 227 | 26 | 10 |
| Agglutination 9 | 86 | 10 | 15 |
| Total | 2326 | 265 | 237 |

In an implementation wherein the system 100 is applied to quantifying the presence of SARS-CoV-2 antibodies, the relationship between antibody titer and level of agglutination (as assigned through the machine learning algorithm 118) is determined by titrating serum from COVID-19 patients. This is followed by computer-based image interpretation. More refined serum titration at 1.5-fold dilution steps showed an approximately linear relationship of antibody titer and neural network training datasets over an 8-fold dilution range, with a two-fold dilution corresponding to a distance of ˜2 neural network training datasets. Together, the data suggest that an image acquisition and computer-based interpretation method can be used to identify hemagglutination. Given the relationship between antibody titer and level of agglutination, the data also suggest that this method can be used to provide a semi-quantitative assessment of antibody titers.

The agglutination identification algorithm described herein may be effectively implemented with testing cards. The testing cards may be constructed of cardboard or a suitable alternative material. The testing cards include two or more regions for code stickers for sample identification and camera focusing. In an implementation, the code stickers include QR codes specific to the test, sample, patient, testing facility, and so forth. Captured image are uploaded by way of a network connection to a server comprising a neural network for performing image analysis.

In an implementation, for quantitative analysis of antibodies, agglutination images are assigned visually (by agreement of one or more human investigators) to 10 arbitrary bins according to the level of agglutination ranging from no agglutination at level 0 to maximum observed agglutination at level 9, separated into sets of training, validation, and holdout labels. The holdout set is composed of an exclusive set of image captures that were not presented to the machine learning algorithm 118 during the training process. The training and validation data sets are used to train a second machine learning algorithm 118 using the same architecture and training procedure as the first machine learning algorithm 118, with the output being a discrete agglutination level for each spot rather than positive or negative identification. Through testing on the expanded holdout set, it was determined that the quantitative model would benefit from a spurious object confidence threshold of 0.5, in contrast to the original arbitrary threshold of 0.2. In addition to filtering spurious predictions by confidence, the standardized layout and geometry of the test card enables valid blood spot predictions to be constrained to appropriate sizes and locations, further enhancing the robustness of the analysis process.

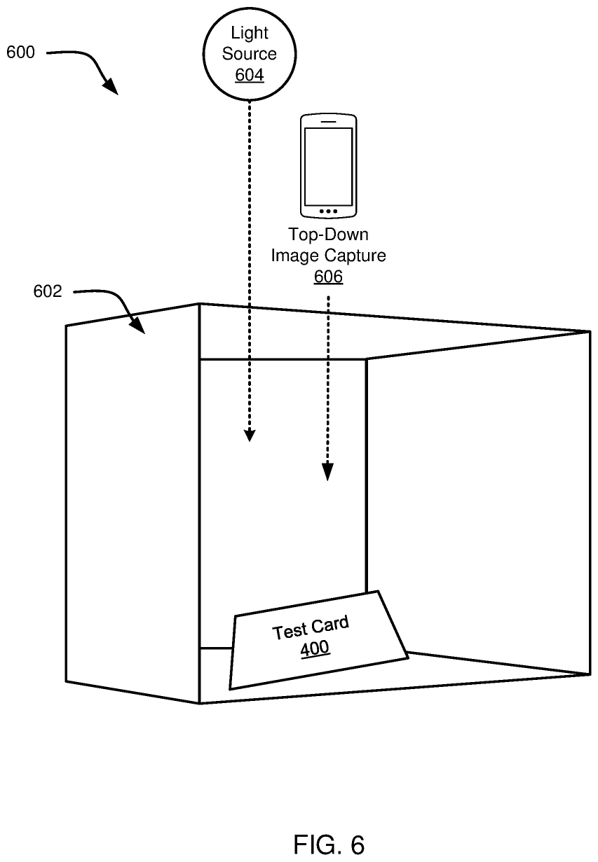

FIG. 6 is a schematic diagram of a system 600 for capturing an image of the test card 400. The system 600 includes light box 602 and a light source 604. The test card 400 is placed within the light box 602 to receive bright and consistent lighting from the light source 604. The system 600 promotes consistent image capture of numerous test cards 400 to increase the likelihood the machine learning algorithm 118 can accurately assess the agglutination of the various blood samples. The temperature and brightness of the light source 604 are selected and optimized for capturing images that can be quickly and accurately assessed by the machine learning algorithm 118.

The system 600 may additionally include a resting place for a camera (may be a mobile phone or tablet) and/or markings indicating where the image sensor should be placed when capturing an image of the test card 400. The system 600 is configured for capturing an evenly exposed top-down image of the test card 400 (see 606).

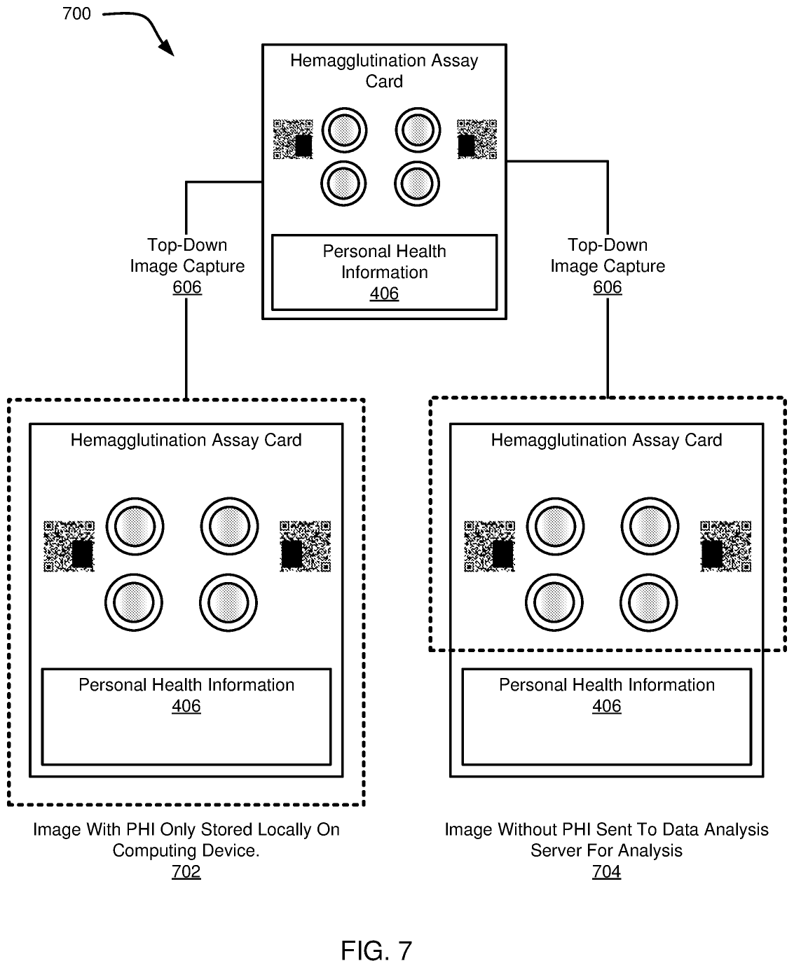

FIG. 7 is a schematic diagram illustrating the partitioning of data across separate storage resources to protect personal health information 406. The test card 400 may include a space for personal health information 406, such as the patient's name, date of birth, contact information, health history information, vaccination information, and so forth. This personal health information 406 is highly confidential and must be treated with care to ensure that outside parties cannot retrieve, for example, the patient's name in connection with the results of the hemagglutination assay card.

In an implementation, an image is taken of the entire test card 400, including the personal health information 406. This image may be stored locally on the computing device 106 that captured the image or another computing device 106 that is hardwired to the image sensor (see 702). This image is not provided to the data analysis server 104 and/or the database 112 by way of the network 110 connection. This significantly reduces the possibility of the personal health information 406 being intercepted or retrieved by an outside party.

The computing device 106 may then transmit an image without the personal health information to the data analysis server 104 and/or the database 112 by way of the network 110 connection (see 704). This image may be a separately captured image that is framed to exclude the personal health information 406 from being captured by the image sensor. Alternatively, this image may be a cropped image of the entire test card 400 to remove the personal health information 406.

The computing device 106 may include a software application configured to capture and upload images of test cards 400 for remote analysis by the machine learning algorithm 118. This software application may be configured to identify and read a lot number and/or serial number printed on the test card 400 and then associate that lot number and/or serial number with the personal health information 406. The lot number and/or serial number is printed on the test card 400 in a region outside the personal health information 406 box, and therefore, the lot number and/or serial number data may be transmitted to the data analysis server 104 and/or the database 112. The computing device 106 locally pairs the personal health information to the lot number and/or serial number. Thus, the machine learning algorithm 118 analyzes the result of the hemagglutination assay without knowing any of the personal health information 406 associated with that blood sample. The machine learning algorithm 118 provides the result of the hemagglutination assay back to the computing device 106 with an indication of the lot number and/or serial number associated with that hemagglutination assay. Then, the computing device 106 looks up the lot number and/or serial number to identify the personal health information 406 (e.g., patient's name, date of birth, and so forth) associated with that hemagglutination assay. This ensures that the patient's personal health information 406 is never transmitted over the network 110 or stored on the database 112 or the data analysis server 104. The patient's personal health information 406 is only stored locally on the computing device 106 that captured the image of the test card 400 (or another computing device that is in local communication with an image sensor that captured the image of the test card 400).

When the result of the hemagglutination assay is again associated with the patient's personal health information 406, then this information may be uploaded to a cloud-based database if needed. The result of the hemagglutination assay may be stored on a remote database such that the patient and healthcare providers can remotely access the patient's hemagglutination result. This information may be encrypted and stored securely such that only those persons with permissions to view the information may access this private healthcare information.

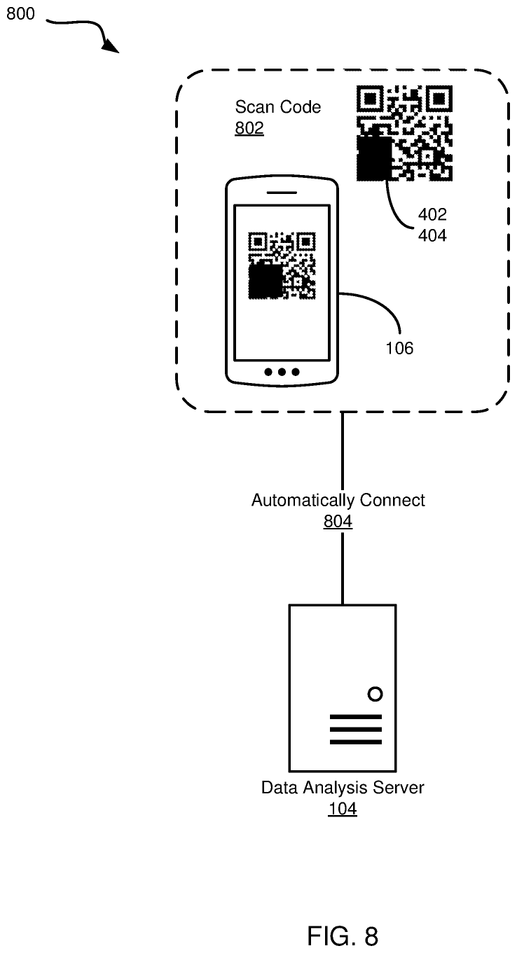

FIG. 8 is a schematic diagram of a system and process flow 800 for communicating with the data analysis server 104 when capturing images of test cards 400 and/or when exchanging data with the data analysis server 104. The process flow 800 may be initiated by a test administrator at a point-of-care location, by a laboratory technician, or by a patient.

In an implementation, the system 600 illustrated in FIG. 6 is present at a point-of-care antibody testing facility. A test administrator retrieves the blood sample from the patient, applies drops of the patient's blood sample to each of the sample spots (see, e.g., 408-414), and then opens a software application on a computing device 106. The software application instructs the test administrator to capture an image of the test card 400. The software application is in communication with an image sensor that may be integrated into the computing device 106 (such as a mobile phone comprising a camera) or may be otherwise in communication with the computing device 106. The software application scans the one or more unique codes 402, 404 on the test card 400 and provides instructions to the test administrator to capture an image of the test card 400 with the appropriate framing, exposure, focus, and so forth. It should be appreciated that this method may alternatively be performed by a technician at a laboratory, by the patient, by a healthcare professional, or by any other individual as needed.

The test administrator (or other individual) then causes the hemagglutination assay on the test card 400 to be transmitted to the data analysis server 104 and/or database 112 by way of the network 110 connection. The machine learning algorithm 118 performs remote image analysis on the hemagglutination assay to qualitatively and/or quantitatively determine the presence of antibodies in the patient's blood sample. The data analysis platform 102 returns the results of the hemagglutination assay in near real-time to the software application on the computing device 106. The results of the hemagglutination assay may then be provided to the patient at the point-of-care location. The results of the hemagglutination assay may additionally be uploaded to a cloud-based database that is made accessible to the patient and/or healthcare providers to view the patient's results.

The one or more unique codes 402, 404 on the test card 400 may be any scannable figure or code that is readable by a device. In an embodiment, the one or more unique codes 402, 404 include a two-dimensional barcode such as a quick response (QR) code. The two-dimensional barcode can be digitally scanned by a camera or other sensor. In an embodiment, the one or more unique codes 402, 404 are designed to appear like a graphic or image and the two-dimensional barcode is integrated or “hidden” in the image. The one or more unique codes 402, 404 may include multiple squares that can be read by the image sensor of the personal device 114.

In an embodiment where the one or more unique codes 402, 404 is a QR code, the code includes three large squares (the three large squares can be seen in the upper-left, lower-left, and upper-right corners of the examples in FIG. 4) that serve as alignment targets while a smaller square in a remaining corner of the one or more unique codes 402, 404 (the smaller square can be seen near the lower-right corner of the example one or more unique codes 402, 404 shown in FIG. 4) serves to normalize the angle with which the image sensor hits the one or more unique codes 402, 404. The remaining area of the one or more unique codes 402, 404 is the actual data that is converted into binary code by the personal device 114. The one or more unique codes 402, 404 may include many characters worth of data. In an example where the one or more unique codes 402, 404 is a 117-pixel square, the code may hold 1852 characters of data.

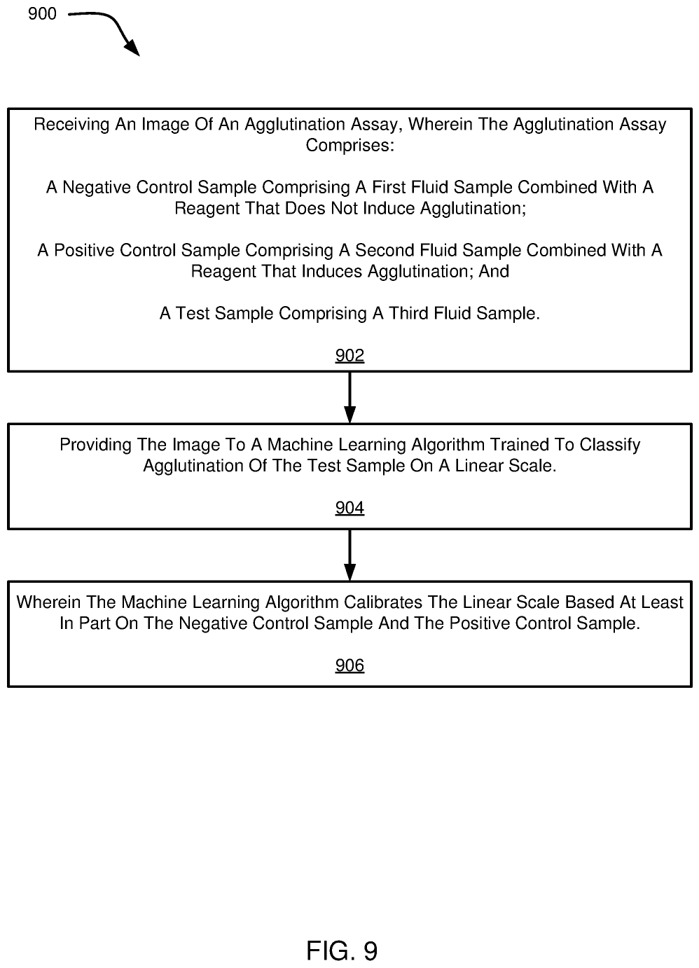

FIG. 9 is a schematic flow chart diagram of a method 900 for computer-implemented image analysis of an agglutination assay. The method 900 includes receiving at 902 an image of an agglutination assay. The agglutination assay includes a negative control sample comprising a first fluid sample combined with a reagent that does not induce agglutination, a positive control sample comprising a second fluid sample combined with a reagent that induces agglutination, and a test sample comprising a third fluid sample. The method 900 includes providing at 904 the image to a neural network trained to classify agglutination of the test sample on a linear scale. The method is such that the neural network calibrates at 906 the linear scale based at least in part on the negative control sample and the positive control sample.

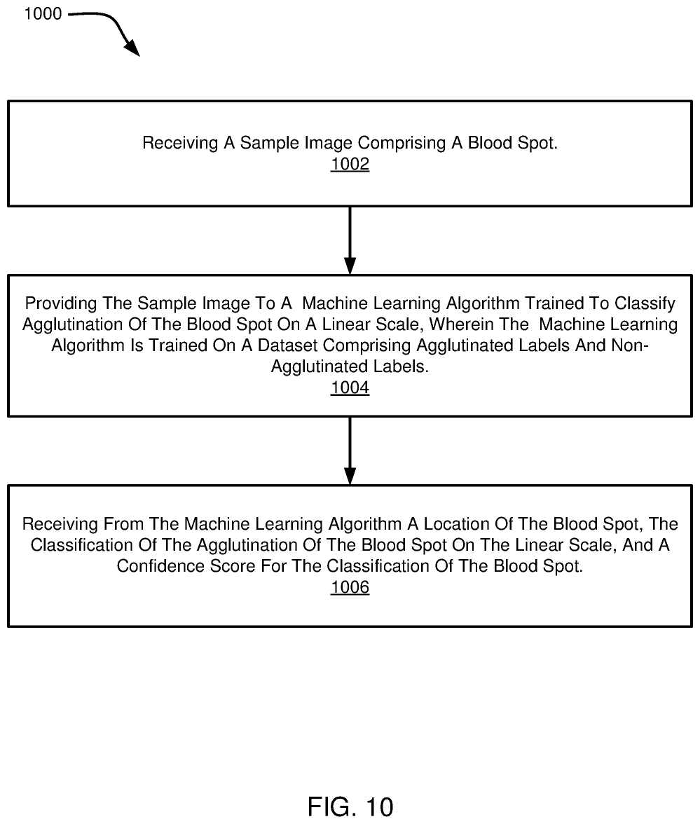

FIG. 10 is a schematic flow chart diagram of a method 1000 for quantifying agglutination of a blood sample. The method 1000 begins and the data analysis server 104 receives at 1002 a sample image comprising a blood spot. The sample image may include an image of the hemagglutination test card 400 captured by a camera 108 or computing device 106. The image comprises a digital capture of one or more blood spots and may further comprise a digital capture of one or more unique codes. The method 1000 continues and the data analysis server 104 provides at 1004 the sample image to a machine learning algorithm 118 trained to classify agglutination of the blood spot in a quantitative manner , wherein the machine learning algorithm 118 is trained on a dataset comprising agglutinated labels and non-agglutinated labels. The network is trained on various levels of agglutination, which is proportional to the concentration of antibodies in the blood sample, thus allowing quantification of antibody levels in patient samples. Quantification is enabled by the positive control 410 and second test 414, which are calibrated based on known antibody standards (e.g., WHO standards with known concentrations of antibodies). The method 1000 continues and the data analysis server 104 receives at 1006 from the machine learning algorithm 118 a location of the blood spot, the classification of the agglutination of the blood spot on the linear scale, and a confidence score for the classification of the blood spot.

The method 1000 may additionally include determining whether the blood spot is agglutinated or non-agglutinated based on the classification of the agglutination of the blood spot that is output by the machine learning algorithm 118. Depending on the implementation and the reagent used to treat the blood spot, this determination of the agglutination of the blood spot may be used to determine whether the user's blood comprises antibodies for a certain virus or other antigen. In one implementation, the blood spot is treated with the reagent described herein for identifying SARS-CoV-2 antibodies within a patient's bloodstream.

The method 1000 may additionally include reading the unique codes present on the hemagglutination test card 400 to determine, for example, an identity of the patient, and identity of the administering laboratory, healthcare facility, or healthcare professional, a type of reagent used to treat the blood sample, a batch number for the reagent used to treat the blood sample, a type of test being performed on the blood sample, and so forth. The method 1000 may additionally include providing instructions to a user by way of a user interface to assist the user in capturing the sample image. The instructions may include, for example, an indication that the user should adjust one or more of lighting, white balance, focus, distance from the blood spot, orientation, and so forth to capture a sample image that can be accurately assessed by the machine learning algorithm 118.

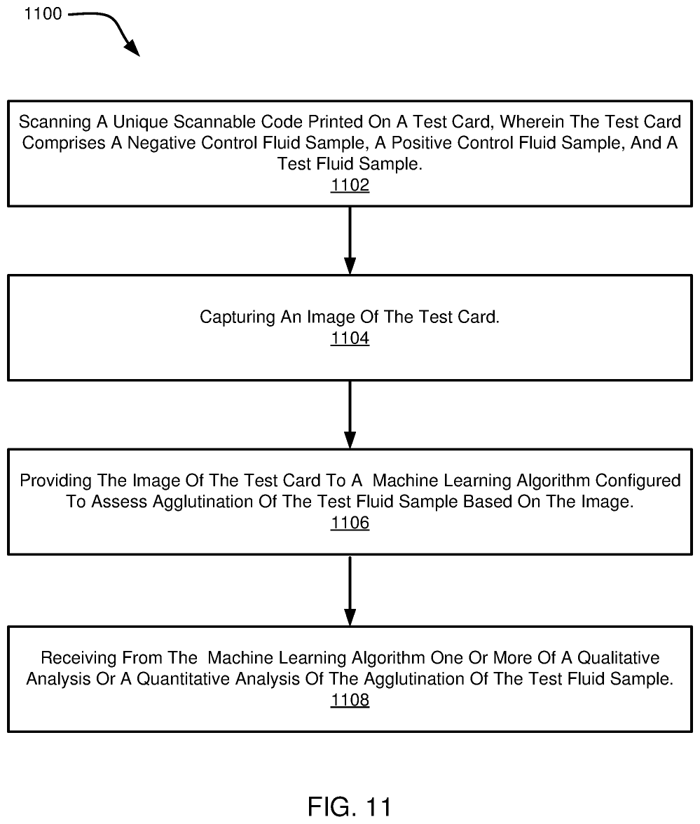

FIG. 11 is a schematic flow chart diagram of a method 1100 for remote image analysis for visually assessing agglutination of fluid samples. The method 1100 includes scanning at 1102 a unique scannable code printed on a test card, wherein the test card comprises a negative control fluid sample, a positive control fluid sample, and a test fluid sample. The method 1100 includes capturing at 1104 an image of the test card. The method 1100 includes providing at 1106 the image of the test card to a neural network configured to assess agglutination of the test fluid sample based on the image. The method 1100 includes receiving at 1108 from the neural network one or more of a qualitative analysis or a quantitative analysis of the agglutination of the test fluid sample.

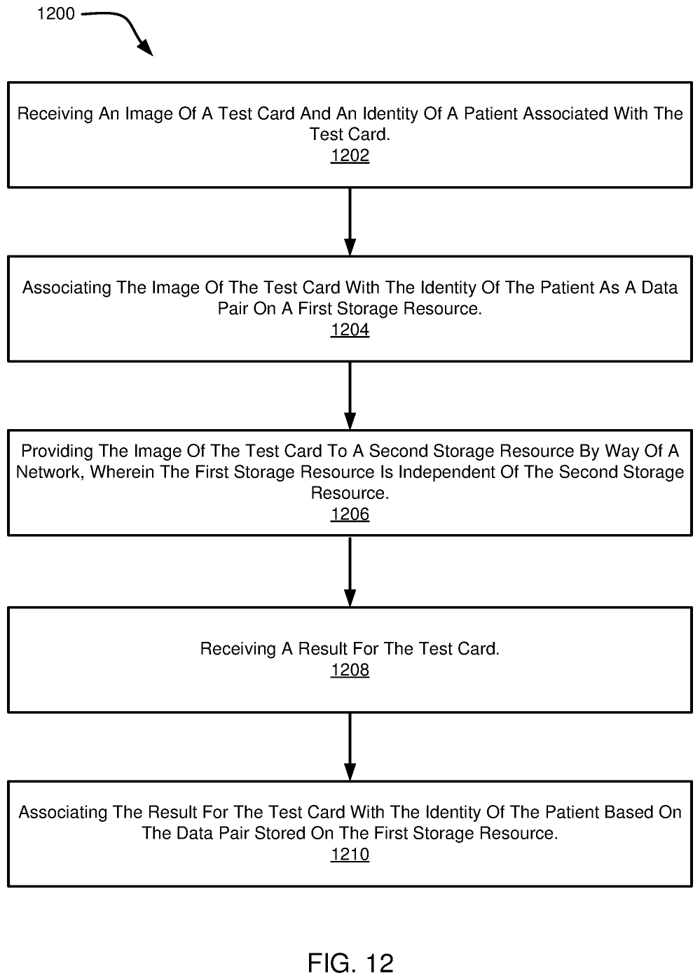

FIG. 12 is a schematic flow chart diagram of a method 1200 for protecting information by partitioning data across multiple storage resources. The method 1200 includes receiving at 1202 an image of a test card and an identity of a patient associated with the test card. The method 1200 includes associating at 1204 the image of the test card with the identity of the patient on a first storage resource. The method 1200 includes providing at 1206 the image of the test card to a second storage resource by way of a network, wherein the first storage resource is independent of the second storage resource. The method 1200 includes receiving at 1208 a result for the test card. The method 1200 includes associating at 1210 the result for the test card with the identity of the patient based on the data pair stored on the first storage resource. Study: Hemagglutination Test For SARS-CoV-2 Specific Antibodies

One implementation of the systems and methods described herein is quantifying SARS-CoV-2-specific antibodies. The SARS-CoV-2 hemagglutination test described herein is based on bispecific proteins that can be used to detect SARS-CoV-2-specific antibodies in finger-stick blood. The simple format, inexpensive reagents, and fast results suggest suitability as a point-of-care test. The data further suggest that machine learning algorithm 118 driven image analysis can be used to identify and quantify hemagglutination on synthetic planar surfaces to determine relative antibody titers.

Further to the above implementation, proteins were expressed in Expi293F cells (ThermoFisher Scientific) using the ExpiFectamine™ 293 Transfection Kit following the manufacturer's instructions (ThermoFisher Scientific). In brief, cells were cultured at 37° C. and 8% CO2 in 25 ml of Expi293 expression medium (ThermoFisher Scientific) in 125 ml bottles on a 25 mm orbital shaker (120 rpm) until reaching a density of 4.5-5.5×10e6/ml. Cells were seeded at 3×10e6/ml in 25 ml per 125ml flasks for transfection. 25 μg of DNA and 80 μl of Epifectamine was added to 1.5 ml and 1.4 ml Opti-Plex complexation buffer (ThermoFisher Scientific), respectively. After a 3-5 min incubation at room temperature (RT), the two solutions were combined, gently mixed, incubated for 15 min at RT and added dropwise to cells. After overnight culture, Epifectamine transfection enhancer 1 (150 μl) and Epifectamine transfection enhancer 2 (1.5 ml) was added, and cell suspensions were harvested two days later.

Cell suspensions were centrifuged at 450 g for 5 min and protein-containing supernatant (SN) was cleared using 0.2 μm filter bottles. To remove biotin (interfering with affinity purification), 3 ml of 10× buffer W (1M Tris-HCl pH 8.0, 1.5 M NaCl, 10 mM EDTA) was added to SN along with 600 μl of BioLock solution (IBA), followed by ultracentrifugation at 20,000 g for 20 min at 4C°. The resulting SN was loaded on columns containing 700 μl of washed Strep-Tactin XT matrix (IBA). The columns were rinsed five times with 1 ml ice-cold PBS, proteins were eluted with 3×500 μl of buffer BXT (IBA), concentrated on Amicon Ultra3k concentration columns (3kD cutoff) at 14,000 g at 4° C. and desalted by centrifugation at 1500 g for 2 min using TBS-equilibrated Zeba Spin desalting columns (0.5 ml, 7 k MWCO, ThermoFisher Scientific). 3-10 μg of purified proteins were analyzed by SDS PAGE and Sypro Ruby staining (ThermoFisher Scientific).

Human de-identified blood and serum samples obtained by venipuncture were used in this study. For whole blood, 10-20 μl of EDTA blood was mixed with an equal volume of the reaction mixture containing recombinant proteins and spread over the test field (˜1 cm diameter) on agglutination cards. For serum, 5-10 μl of serum were added to the same volume of washed RBC of blood group 0. An equivalent volume of the reaction mixture containing recombinant proteins were added and treated as described for whole blood. The mixed sample was swiveled manually, and images were taken during time using a cell phone camera.

Specimens were tested using the Abbott, Siemens and EUROIMMUN SARS-CoV-2 IgG EUA cleared immunoassays. The Abbott SARS-CoV-2 IgG assay was performed on the Abbott Architect i2000 (Abbott Laboratories Inc, Abbott Park, Ill.) according to the manufacturer's instructions. This is a qualitative chemiluminescent microparticle immunoassay (CMIA) that detects IgG to the Nucleocapsid protein of SARS-CoV-2. According to the manufacturer, there is a direct relationship between the amount of SARS-CoV-2 specific IgG in the sample and the calculated signal of the sample divided by the signal of the calibrator (S/C) index. A result is considered positive if the S/C is greater than or equal to 1.4. The Siemens Healthcare Diagnostics ADVIA Centaur SARS-CoV-2 IgG (COV2G) was performed on the ADVIA Centaur XPT Immunoassay System (Siemens Healthcare Diagnostics Inc. Tarrytown, NY) according to the manufacturer's instructions. This is a semi-quantitative high-throughput chemiluminescent immunoassay (CLIA) that detects IgG to the 51 RBD region of SARS-CoV-2. According to the manufacturer, there is a direct relationship between the amount of SARS-CoV-2 antibody present in the patient sample and the amount of relative light units (RLU) detected by the system. A result of reactive or nonreactive is determined according to the Index Value established with the kit calibrators. A sample is considered reactive or positive for S1 RBD IgG when the Index is >1.00. The EUROIMMUN assay was performed manually according to the manufacturer's instructions using the Anti-SARS-CoV-2 IgG Enzyme Linked Immunosorbent Assay (EUROIMMUN US, Mountain Lakes, NJ). The assay format is a 96-well microtiter plate coated with SARS-CoV-2 recombinant S1 spike protein produced in HEK 293 cells. Results are calculated as the ratio between the OD of the sample and the OD of the calibrator. This sample to calibrator ratio is interpreted as negative when the ratio is below 0.8, borderline if the ratio is between 0.8 and 1.1, and positive if the ratio is greater than or equal to 1.1. All results were interpreted according to the manufacturer's cutoffs.

The S-Flow assay was performed as previously described with minor modifications. Briefly, 293T cells were transiently transfected with an expression vectors for SARS-CoV-2 Spike or control vector. Two days after transfection, cells were harvested and incubated with patient sera diluted 1:625 in Facs buffer (PBS with 0.5% BSA and 2 mM EDTA) for 20 minutes at 4° C. After washing, cells were stained with goat anti-human IgG Alexa Fluor 647 (1:200; Thermo Fisher Scientific) and goat anti-human IgM Alexa Fluor 488 (1:200; Thermo Fisher Scientific) for 20 minutes at 4° C. Cells were washed, fixed in 4% formaldehyde, and analyzed on a BD LSRFortessa flow cytometer for antibody binding. Specific antibody binding was determined based on control-vector transfected cells. Serum was considered test-positive if the percentage of IgG-positive cells of Spike-transfected cells exceeded the percentage of control-vector transfected cells by more than 10%.

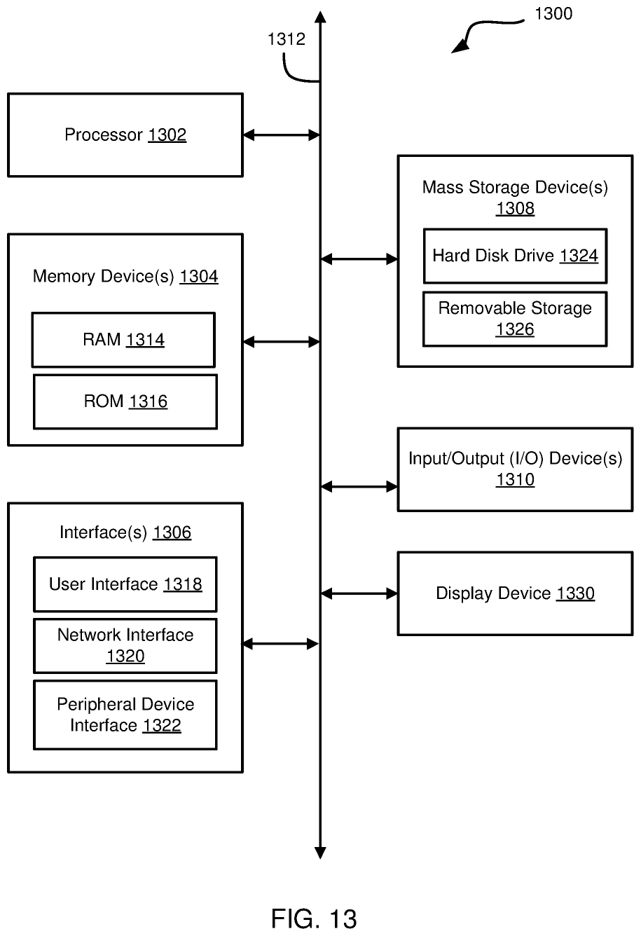

Referring now to FIG. 13, a block diagram of an example computing device 1300 is illustrated. Computing device 1300 may be used to perform various procedures, such as those discussed herein. Computing device 1300 can perform various monitoring functions as discussed herein, and can execute one or more application programs, such as the application programs or functionality described herein. Computing device 1300 can be any of a wide variety of computing devices, such as a desktop computer, in-dash computer, vehicle control system, a notebook computer, a server computer, a handheld computer, tablet computer and the like.

Computing device 1300 includes one or more processor(s) 1302, one or more memory device(s) 1304, one or more interface(s) 1306, one or more mass storage device(s) 1308, one or more Input/output (I/O) device(s) 1310, and a display device 1330 all of which are coupled to a bus 1312. Processor(s) 1302 include one or more processors or controllers that execute instructions stored in memory device(s) 1304 and/or mass storage device(s) 1308. Processor(s) 1302 may also include various types of computer-readable media, such as cache memory.

Memory device(s) 1304 include various computer-readable media, such as volatile memory (e.g., random access memory (RAM) 1314) and/or nonvolatile memory (e.g., read-only memory (ROM) 1316). Memory device(s) 1304 may also include rewritable ROM, such as Flash memory.

Mass storage device(s) 1308 include various computer readable media, such as magnetic tapes, magnetic disks, optical disks, solid-state memory (e.g., Flash memory), and so forth. As shown in FIG. 13, a particular mass storage device 1308 is a hard disk drive 1324. Various drives may also be included in mass storage device(s) 1308 to enable reading from and/or writing to the various computer readable media. Mass storage device(s) 1308 include removable media 1326 and/or non-removable media.

I/O device(s) 1310 include various devices that allow data and/or other information to be input to or retrieved from computing device 1300. Example I/O device(s) 1310 include cursor control devices, keyboards, keypads, microphones, monitors or other display devices, speakers, printers, network interface cards, modems, barcode scanners, and the like.

Display device 1330 includes any type of device capable of displaying information to one or more users of computing device 1300. Examples of display device 1330 include a monitor, display terminal, video projection device, and the like.

Interface(s) 1306 include various interfaces that allow computing device 1300 to interact with other systems, devices, or computing environments. Example interface(s) 1306 may include any number of different network interfaces 1320, such as interfaces to local area networks (LANs), wide area networks (WANs), wireless networks, and the Internet. Other interface(s) include user interface 1318 and peripheral device interface 1322. The interface(s) 1306 may also include one or more user interface elements 1318. The interface(s) 1306 may also include one or more peripheral interfaces 1322 such as interfaces for printers, pointing devices (mice, track pad, or any suitable user interface now known to those of ordinary skill in the field, or later discovered), keyboards, and the like.

Bus 1312 allows processor(s) 1302, memory device(s) 1304, interface(s) 1306, mass storage device(s) 1308, and I/O device(s) 1310 to communicate with one another, as well as other devices or components coupled to bus 1312. Bus 1312 represents one or more of several types of bus structures, such as a system bus, PCI bus, IEEE bus, USB bus, and so forth.

### EXAMPLES

The following examples pertain to further embodiments.

Example 1 is a method. The method includes receiving a sample image comprising a blood spot. The method includes providing the sample image to a neural network trained to classify agglutination of the blood spot on a linear scale, wherein the neural network is trained on a dataset comprising agglutinated labels and non-agglutinated labels. The method includes receiving from the neural network a location of the blood spot, the classification of the agglutination of the blood spot on the linear scale, and a confidence score for the classification.

Example 2 is a method as in Example 1, wherein the blood spot captured in the sample image comprises one or more droplets of blood retrieved from a user by way of one or more of a fingerstick sampling, venipuncture sampling, or arterial sampling.

Example 3 is a method as in any of Examples 1-2, wherein the blood spot captured in the sample image comprises one or more droplets of blood retrieved from a user for a viral hemagglutination assay.

Example 4 is a method as in any of Examples 1-3, further comprising receiving from the neural network a quantification of viral antibodies based on the viral hemagglutination assay.

Example 5 is a method as in any of Examples 1-4, wherein the viral hemagglutination assay is implemented for quantifying a presence of SARS-CoV-2 antibodies in the user.

Example 6 is a method as in any of Examples 1-5, wherein the viral hemagglutination assay is implemented to quantify a relationship between antibody titer and level of agglutination by titrating serum from the user.

Example 7 is a method as in any of Examples 1-6, wherein the sample image further comprises a testing card, wherein the blood spot is deposited on the testing card, and wherein the testing card and blood spot are placed in light box prior to capturing the sample image.

Example 8 is a method as in any of Examples 1-7, wherein the sample image is captured by a sample image sensor associated with a mobile phone.

Example 9 is a method as in any of Examples 1-8, wherein receiving the sample image comprises receiving the sample image over a network connection, and wherein the method further comprises providing the prediction to the mobile phone over the network connection such that the viral hemagglutination assay can be provided as point-of-care results to the user.

Example 10 is a method as in any of Examples 1-9, wherein the neural network is further configured to quantify an agglutination of the blood spot based on a spurious object confidence threshold of 0.5.

Example 11 is a method as in any of Examples 1-10, wherein the viral hemagglutination assay is implemented at least in part by applying bispecific proteins for detecting SARS-CoV-2-specific antibodies in finger-stick blood retrieved from the user.

Example 12 is a method as in any of Examples 1-11, wherein the classification of the agglutination of the blood spot comprises a quantified likelihood of agglutination, wherein the likelihood of agglutination is calculated based on the linear scale.

Example 13 is a method as in any of Examples 1-12, wherein the linear scale comprises a scale from 0 to 10, and wherein the neural network classifies the agglutination of the blood spot based on the linear scale, and wherein the method further comprises determining whether the blood spot is agglutinated or non-agglutinated based on the classification on the linear scale.

Example 14 is a method as in any of Examples 1-13, wherein a classification from 0 to 1 on the linear scale indicates the blood spot is non-agglutinated.

Example 15 is a method as in any of Examples 1-14, wherein a classification from 7 to 10 on the linear scale indicates the blood spot is agglutinated.

Example 16 is a method as in any of Examples 1-15, further comprising providing a positive image comprising an agglutinated blood spot sample to the neural network and a providing negative image comprising a non-agglutinated blood spot sample to the neural network.

Example 17 is a method as in any of Examples 1-16, wherein the neural network is configured to classify the agglutination of the blood spot on the linear or non-linear (discrete or continouous) scale at least in part by comparing the sample image with the positive image and the negative image.

Example 18 is a method as in any of Examples 1-17, wherein the image is captured by an image sensor associated with a mobile phone as part of a point-of-care assessment for determining whether the user comprises antibodies for SARS-CoV-2.

Example 19 is a method as in any of Examples 1-18, further comprising determining whether the blood spot is agglutinated or non-agglutinated based on the classification of the agglutination of the blood spot on the linear scale.

Example 20 is a method as in any of Examples 1-19, wherein the blood spot captured in the image is treated with a reagent for identifying a presence of SARS-CoV-2 virus, and wherein the method further comprises determining whether the user comprises antibodies for the SARS-CoV-2 virus based on the classification of the agglutination of the blood spot on the linear or non-linear scale.

Example 21 is a method as in any of Examples 1-20, further comprises providing an instruction to a user by way of a user interface indicating the user should capture the image comprising the blood spot.

Example 22 is a method as in any of Examples 1-21, wherein providing the instruction to the user to capture the image further comprises providing the instruction in response to determining the blood spot is lit for processing by the neural network to classify the agglutination of the blood spot.

Example 23 is a method. The method includes receiving an image of an agglutination assay, wherein the agglutination assay comprises: a negative control sample comprising a first fluid sample combined with a reagent that does not induce agglutination; positive control samples comprising fluid samples combined with reagents that induce agglutination; and a test sample comprising a third fluid sample. The method includes providing the image to a neural network trained to classify agglutination of the test sample on a linear scale. The method is such that the neural network calibrates the linear scale based at least in part on the negative control sample and the positive control sample.

Example 24 is a method as in Example 23, wherein the image of the agglutination assay comprises an image of a test card comprising: a negative control testing region, wherein the first fluid sample and the reagent that does not induce agglutination are deposited on the test card within the negative control testing region; positive control testing regions, wherein fluid samples and the reagent that induces agglutination are deposited on the test card within the positive control testing region; a test sample testing region, wherein the third fluid sample is deposited on the test card within the test sample testing region; and a unique code that is scannable by a computing device.

Example 25 is a method as in any of Examples 23-24, wherein each of the test sample testing region further comprises a recombinant protein deposited on the test card within the test sample testing region, and wherein the recombinant protein is configured to mediate binding of red blood cells to a component of an antigen or a byproduct of the antigen.

Example 26 is a method as in any of Examples 23-25, wherein the antigen is the SARS-CoV-2 virus, and wherein the recombinant protein comprises a nanobody that mediates the binding of red blood cells within the third fluid sample to one or more of: a receptor-binding domain of a spike protein associated with the SARS-CoV-2 virus; or a nucleocapsid protein associated with the SARS-CoV-2 virus.

Example 27 is a method as in any of Examples 23-26, wherein classifying the agglutination of the test sample on the linear or non-linear scale comprises determining whether the third fluid sample comprises antibodies for an identified antigen.

Example 28 is a method as in any of Examples 23-27, wherein classifying the agglutination of the test sample on the linear or non-linear scale comprises quantifying a presence of antibodies within the third fluid sample; wherein the antibodies are generated in response to an identified antigen; and wherein the test sample further comprises a recombinant protein configured to mediate binding of red blood cells within the third fluid sample to the identified antigen or a byproduct of the identified antigen.

Example 29 is a method as in any of Examples 23-28, wherein each of the first fluid sample, the second fluid sample, and the third fluid sample constitutes an agglutination sample retrieved from a patient in a single session, and wherein the neural network is further configured to output a test result for the patient comprising one or more of: an indication that no antibodies for the identified antigen were identified in the patient's agglutination sample; an indication that the antibodies for the identified antigen were identified in the patient's agglutination sample; or a quantified result on the linear scale indicating a degree to which the antibodies for the identified antigen were identified in the patient's agglutination sample.

Example 30 is a method as in any of Examples 23-29, wherein the output comprising the test result further comprises a qualitative result indicating a degree to which the antibodies for the identified antigen were identified in the patient's agglutination sample.

Example 31 is a method as in any of Examples 23-30, wherein the qualitative result comprises one or more of: the indication that no antibodies for the identified antigen were identified in the patient's agglutination sample; an indication that a low quantity of antibodies for the identified antigen were identified in the patient's agglutination sample; an indication that a moderate quantity of antibodies for the identified antigen were identified in the patient's agglutination sample; or an indication that a high quantity of antibodies for the identified antigen were identified in the patient's agglutination sample.

Example 32 is a method as in any of Examples 23-31, wherein the output comprising the test result further comprises an estimation of whether the patient has been exposed to the identified antigen.

Example 33 is a method as in any of Examples 23-32, wherein the output comprising the test result further comprises an estimation of whether the patient has been vaccinated against the identified antigen.

Example 34 is a method as in any of Examples 23-33, wherein each of the first fluid sample, the second fluid sample, and the third fluid sample is a blood sample retrieved from a patient in a single session.

Example 35 is a method as in any of Examples 23-34, wherein the blood sample is retrieved from the patient by way of a fingerstick sampling, and wherein one or more drops of blood from the fingerstick sampling are deposited directly on to a test card for the agglutination assay for each of the negative control sample, the positive control sample, and the test sample.

Example 36 is a method as in any of Examples 23-35, wherein the neural network is trained on a dataset comprising images with agglutinated labels and images with non-agglutinated labels.

Example 37 is a method as in any of Examples 23-36, wherein the neural network is trained on a dataset comprising: a plurality of images of blood samples from patients who have not been infected with an identified antigen and who have not been vaccinated against the identified antigen; a plurality of images of blood samples from patients who have not been infected with the identified antigen and have been vaccinated against the identified antigen; a plurality of images of blood samples from patients who have been infected with the identified antigen and have not been vaccinated against the identified antigen; and a plurality of images of blood samples from patients who have been infected with the identified antigen and have been vaccinated against the identified antigen.

Example 38 is a method as in any of Examples 23-37, wherein the neural network calibrates the linear scale based on an appearance of: the negative control sample comprising a reaction of the first fluid sample with the reagent that does not induce agglutination; and the positive control sample comprising a reaction of the second fluid sample with the reagent that induces agglutination.

Example 39 is a method as in any of Examples 23-38, wherein the neural network calibrates the linear scale by measuring a relative difference between the negative control sample and the positive control sample to identify natural background agglutination for a patient that provided the first fluid sample, the second fluid sample, and the third fluid sample.

Example 40 is a method as in any of Examples 23-39, wherein the neural network classifies the agglutination of the test sample in view of the natural background agglutination for the patient.

Example 41 is a method as in any of Examples 23-40, wherein: the appearance of the negative control sample represents zero agglutination for the patient; the appearance of the positive control sample represents maximum agglutination for the patient; and the appearance of the test sample represents relative agglutination for the patient in a presence of a recombinant protein based on the linear scale from the zero agglutination to the maximum agglutination.

Example 42 is a method as in any of Examples 23-41, wherein the recombinant protein is a hemagglutination-based bispecific protein comprising a nanobody that binds red blood cells and an identified antigen or a byproduct of the identified antigen.

Example 43 is a method as in any of Examples 23-42, wherein the neural network is configured to identify one or more objects of interest within the image of the agglutination assay and draw a bounding box around each of the one or more objects of interest, and wherein the one or more objects of interest comprise one or more of: a region comprising negative control sample; a region comprising the positive control sample; or a region comprising the test sample.

Example 44 is a method as in any of Examples 23-43, wherein the neural network is trained to calibrate the linear scale based on a training dataset, and wherein the training dataset comprises a plurality of agglutination images that have been classified with a quantified agglutination level on a scale ranging from no agglutination to maximum agglutination.

Example 45 is a test card. The test card includes a negative control test region for receiving a negative control fluid sample, positive control test regions for receiving positive control fluid samples, and a test sample region for receiving a test fluid sample. The test card includes one or more unique scannable codes comprising data for instructing a processor to capture an image of the test card that is suitable for computer-implemented image analysis.

Example 46 is a test card as in Example 45, wherein the negative control test region comprises a negative reagent that does not mediate agglutination of the negative control fluid sample; and positive control test regions comprising positive reagents that cause agglutination of the positive control fluid samples.

Example 47 is a test card as in any of Examples 45-46, wherein the test sample region comprises a test reagent that mediates binding of red blood cells within the test fluid sample to an identified antigen or a byproduct of the identified antigen.

Example 48 is a test card as in any of Examples 45-47, wherein each of the negative control fluid sample, the positive control fluid sample, and the test fluid sample is a blood sample retrieved from a patient.

Example 49 is a test card as in any of Examples 45-48, wherein each of the negative reagent, the positive reagent, and the test reagent is activated by a fingerstick sampling blood sample retrieved from a patient.

Example 50 is a test card as in any of Examples 45-49, wherein the test card is an agglutination assay for assessing a presence of antibodies for SARS-CoV-2 virus in a blood sample.

Example 51 is a test card as in any of Examples 45-50, wherein the positive control test region comprises a positive reagent that comprises a nanobody configured to mimic a function of physiological antibodies directed against a protein associated with the SARS-CoV-2 virus to induce hemagglutination of the positive control fluid sample.

Example 52 is a test card as in any of Examples 45-51, wherein the test sample region comprises a test reagent that comprises a nanobody configured to induce hemagglutination of the test fluid sample if the test fluid sample comprises antibodies for the SARS-CoV-2 virus.

Example 53 is a test card as in any of Examples 45-52, wherein the test sample region comprises a test reagent that is based on bispecific proteins comprising a dromedary-derived antibody configured to bind red blood cells in the test fluid sample and an antigen derived from the SARS-CoV-2 virus.

Example 54 is a test card as in any of Examples 45-53, wherein the antigen derived from the SARS-CoV-2 virus comprises one or more of a receptor-binding coming of a spike protein associated with the SARS-CoV-2 virus or a nucleocapsid protein.

Example 55 is a test card as in any of Examples 45-54, wherein the test card comprises two or more unique scannable codes comprising: a first unique scannable code comprising data pertaining to the test card; and a second unique scannable code comprising the data for instructing the processor to capture the image of the test card that is suitable for the computer-implemented image analysis.

Example 56 is a test card as in any of Examples 45-55, wherein the test card comprises two or more unique scannable codes, and wherein each of the two or more unique scannable codes comprises: data pertaining to the test card that identifies one or more of a lot number or a serial number associated with the test card; and the data for instructing the processor capture the image of the test card that is suitable for computer-implemented image analysis.

Example 57 is a test card as in any of Examples 45-56, wherein the data for instructing the processor to capture the image of the test card comprises one or more of: instructions for capturing the image of the test card when an image sensor is positioned with a desired framing of the test card; instructions for capturing the image of the test card when the image sensor is focused on the test card with a sufficient focus for executing the computer-implemented image analysis of the image of the test card; or instructions for capturing the image of the test card when a resultant image will be sufficiently exposed for executing the computer-implemented image analysis of the image of the test card.

Example 58 is a test card as in any of Examples 45-57, wherein the one or more unique scannable codes further comprises data pertaining to the test card, and wherein the data pertaining to the test card comprises one or more of a lot number or a unique serial number associated with the test card.

Example 59 is a test card as in any of Examples 45-58, wherein the one or more unique scannable codes is a Quick Response (QR) code.

Example 60 is a test card as in any of Examples 45-59, wherein the one or more unique scannable codes are readable by a processor in communication with an image sensor.

Example 61 is a test card as in any of Examples 45-60, wherein the computer-implemented image analysis comprises providing the image of the test card to a neural network trained to assess agglutination of the test fluid sample.

Example 62 is a test card as in any of Examples 45-61, wherein the neural network is trained to assess the agglutination of the test fluid sample qualitatively and quantitatively.

Example 63 is a test card as in any of Examples 45-62, wherein the neural network is trained to calibrate a linear scale for qualitatively and quantitatively assessing the agglutination of the test fluid sample, and wherein the linear scale is calibrated based on agglutination of the negative control fluid sample and the positive control fluid sample.

Example 64 is a method. The method includes scanning a unique scannable code printed on a test card, wherein the test card comprises a negative control fluid sample, a positive control fluid sample, and a test fluid sample. The method includes capturing an image of the test card. The method includes providing the image of the test card to a neural network configured to assess agglutination of the test fluid sample based on the image. The method includes receiving from the neural network one or more of a qualitative analysis or a quantitative analysis of the agglutination of the test fluid sample.

Example 65 is a method as in Example 64, wherein the neural network is configured to assess the agglutination of the test fluid sample on a linear scale, and wherein the neural network calibrates the linear scale based on agglutination of each of the negative control fluid sample and the positive control fluid sample.

Example 66 is a method as in any of Examples 64-65, wherein capturing the image of the test card comprises reading out data from a pixel array in electronic communication with a processor of a computing device, and wherein the neural network is executed by a remote computing resource in communication with the computing device by way of a network.

Example 67 is a method as in any of Examples 64-66, wherein: the negative control fluid sample comprises a negative control blood sample in combination with a negative reagent that prevents hemagglutination of the negative control blood sample; the positive control fluid sample comprises a positive control blood sample in combination with a positive reagent that induces maximum hemagglutination of the positive control blood sample; and the test fluid sample comprises a test blood sample in combination with a test reagent that binds red blood cells of the test blood sample with an antigen or a byproduct of the antigen.

Example 68 is a method as in any of Examples 64-67, wherein the neural network is configured to determine whether the test blood sample comprises antibodies for the antigen based on visual hemagglutination of the negative control fluid sample, the positive control fluid sample, and the test fluid sample as depicted in the image of the test card.

Example 69 is a method as in any of Examples 64-68, further comprising: identifying one or more of a lot number or a serial number associated with the test card based on data associated within the unique scannable code; identifying a patient associated with the test card; and storing the one or more of the lot number or the serial number in connection with the patient.

Example 70 is a method as in any of Examples 64-69, further comprising calculating a result indicating whether the patient comprises antibodies for an antigen based on the one or more of the qualitative analysis or the quantitative analysis of the agglutination of the test fluid sample as received from the neural network.

Example 71 is a method as in any of Examples 64-70, wherein scanning the unique scannable code comprises receiving instructions indicating one or more of a desired exposure, framing, or focus for capturing the image of the test card.

Example 72 is a method as in any of Examples 64-71, wherein capturing the image of the test card comprises one or more of: verifying whether the image of the test card will comprise a desired framing such that certain portions of the test card are contained with the image of the test card; verifying whether the image of the test card will comprise suitable focus for executing computer-implemented image analysis with the neural network; or verifying whether the image of the test card will comprise sufficient exposure for executing the computer-implemented image analysis with the neural network.

Example 73 is a method as in any of Examples 64-72, wherein capturing the image of the test card comprises initiating a countdown timer that notifies a user when the image of the test card should be captured to produce a valid result, and wherein the valid result comprises one or more of a desirable exposure, framing, or focus suitable for executing computer-implemented image analysis of the image of the test card with the neural network.

Example 72 is a method. The method includes receiving an image of a test card and an identity of a patient associated with the test card. The method includes associating the image of the test card with the identity of the patient on a first storage resource. The method includes providing the image of the test card to a second storage resource by way of a network, wherein the first storage resource is independent of the second storage resource. The method includes receiving a result for the test card. The method includes associating the result for the test card with the identity of the patient based on the data pair stored on the first storage resource.

Example 73 is a method as in Example 72, wherein the first storage resource is disposed locally on a computing device, and wherein the computing device is in direct electrical communication with an image sensor that captured the image of the test card.

Example 74 is a method as in any of Examples 72-73, wherein the identity of the patient is not provided to the second storage resource.

Example 75 is a method as in any of Examples 72-74, wherein the test card comprises an agglutination assay, and wherein receiving the result for the test card comprises receiving one or more of a qualitative or a quantitative analysis of a degree of agglutination of a test sample on the test card.

Example 76 is a method as in any of Examples 72-75, wherein receiving the result for the test card further comprises receiving a linear scale of agglutination that is calibrated for the patient, and wherein the linear scale is determined based on visual agglutination of a negative control sample and a positive control sample on the test card.

Example 77 is a method as in any of Examples 72-76, further comprising: identifying a unique identifier associated with the test card, wherein the unique identifier comprises one or more of a lot number or a serial number; storing the unique identifier in association with the image of the test card and the identity of the patient on the first storage resource; wherein providing the image of the test card to the second storage resource comprises further providing the unique identifier to the second storage resource.

Example 78 is a method as in any of Examples 72-77, wherein: receiving the result for the test card further comprises receiving the unique identifier in connection with the result for the test card; and associating the result for the test card with the identity of the patient comprises associating based on the unique identifier.

Example 79 is a method as in any of Examples 72-78, wherein the identity of the patient comprises personal health information comprising one or more of a patient name, a patient date of birth, a patient contact information, a patient vaccination status, or a patient infection status.

Example 80 is a method as in any of Examples 72-79, wherein the second storage resource is a server comprising one or more processors configured to execute a neural network for performing image analysis on the image of the test card, and wherein receiving the result for the test card comprises receiving an output from the neural network.

Example 81 is a method as in any of Examples 72-80, wherein associating the image of the test card with the identity of the patient on the first storage resource comprises writing metadata to each of the image of the test card and the identity of the patient to indicate that the test card is associated with the patient.

Reference throughout this specification to “an example” means that a particular feature, structure, or characteristic described in connection with the example is included in at least one embodiment of the present disclosure. Thus, appearances of the phrase “in an example” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on its presentation in a common group without indications to the contrary. In addition, various embodiments and examples of the present disclosure may be referred to herein along with alternatives for the various components thereof. It is understood that such embodiments, examples, and alternatives are not to be construed as de facto equivalents of one another but are to be considered as separate and autonomous representations of the present disclosure.

Although the foregoing has been described in some detail for purposes of clarity, it will be apparent that certain changes and modifications may be made without departing from the principles thereof. It should be noted that there are many alternative ways of implementing both the processes and apparatuses described herein. Accordingly, the present embodiments are to be considered illustrative and not restrictive.

Those having skill in the art will appreciate that many changes may be made to the details of the above-described embodiments without departing from the underlying principles of the disclosure. The scope of the present disclosure should, therefore, be determined only by the claims, if any.

What is claimed is:

1. A test card comprising:
a negative control test region for receiving a negative control fluid sample;
a positive control test region for receiving a positive control fluid sample;
a test sample region for receiving a test fluid sample; and
one or more unique scannable codes comprising data for instructing a processor to capture an image of the test card that is suitable for computer-implemented image analysis.

2. The test card of claim 1, wherein:
the negative control test region comprises a negative reagent that prevents agglutination of the negative control fluid sample; and
the positive control test region comprises a positive reagent that causes various levels of agglutination of the positive control fluid sample.

3. The test card of claim 2, wherein the test sample region comprises a test reagent that mediates binding of red blood cells and further comprises an identified antigen or a byproduct of the identified antigen.

4. The test card of claim 3, wherein each of the negative control fluid sample, the positive control fluid sample, and the test fluid sample is a blood sample retrieved from a patient.

5. The test card of claim 3, wherein each of the negative reagent, the positive reagent, and the test reagent is activated by a blood sample retrieved from a patient, wherein the blood sample is retrieved by way of one or more of a fingerstick sampling, a venipuncture sampling, or an arterial sampling.

6. The test card of claim 1, wherein the test card is an agglutination assay for assessing a presence of antibodies for SARS-CoV-2 virus in a blood sample.

7. The test card of claim 6, wherein the negative control test region comprises a negative reagent that comprises a nanobody configured to bind red blood cells and cause non-antigen-specific agglutination of the red blood cells.

8. The test card of claim 6, wherein the positive control test region comprises a positive reagent that comprises a nanobody configured to mimic a function of physiological antibodies directed against a protein associated with the SARS-CoV-2 virus to induce hemagglutination of the positive control fluid sample.

9. The test card of claim 6, wherein the test sample region comprises a test reagent that comprises a nanobody configured to induce hemagglutination of the test fluid sample if the test fluid sample comprises antibodies for the SARS-CoV-2 virus.

10. The test card of claim 6, wherein the test sample region comprises a test reagent that is based on bispecific proteins comprising a dromedary-derived antibody configured to bind red blood cells and further comprises the test fluid sample with an antigen derived from the SARS-CoV-2 virus.

11. The test card of claim 10, wherein the antigen derived from the SARS-CoV-2 virus comprises one or more of a receptor-binding coming of a spike protein associated with the SARS-CoV-2 virus or a nucleocapsid protein.

12. The test card of claim 1, wherein the test card comprises two or more unique scannable codes comprising:
a first unique scannable code comprising data pertaining to the test card; and
a second unique scannable code comprising the data for instructing the processor to capture the image of the test card that is suitable for the computer-implemented image analysis.

13. The test card of claim 1, wherein the test card comprises two or more unique scannable codes, and wherein each of the two or more unique scannable codes comprises:
data pertaining to the test card that identifies one or more of a lot number or a serial number associated with the test card; and
the data for instructing the processor capture the image of the test card that is suitable for computer-implemented image analysis.

14. The test card of claim 1, wherein the data for instructing the processor to capture the image of the test card comprises one or more of:
instructions for capturing the image of the test card when an image sensor is positioned with a desired framing of the test card;
instructions for capturing the image of the test card when the image sensor is focused on the test card with a sufficient focus for executing the computer-implemented image analysis of the image of the test card; or
instructions for capturing the image of the test card when a resultant image will be sufficiently exposed for executing the computer-implemented image analysis of the image of the test card.

15. The test card of claim 1, wherein the one or more unique scannable codes further comprises data pertaining to the test card, and wherein the data pertaining to the test card comprises one or more of a lot number or a unique serial number associated with the test card.

16. The test card of claim 1, wherein the one or more unique scannable codes is a Quick Response (QR) code.

17. The test card of claim 1, wherein the one or more unique scannable codes are readable by a processor in communication with an image sensor.

18. The test card of claim 1, wherein the computer-implemented image analysis comprises providing the image of the test card to a machine learning algorithm trained to assess agglutination of the test fluid sample.

19. The test card of claim 18, wherein the machine learning algorithm is trained to assess the agglutination of the test fluid sample qualitatively and quantitatively.

20. The test card of claim 19, wherein the machine learning algorithm is trained to calibrate a linear or non-linear scale for qualitatively and quantitatively assessing the agglutination of the test fluid sample, and wherein the linear or non-linear scale is calibrated based on agglutination of the negative control fluid sample and the positive control fluid sample.
